SEQUENCE LISTING (15 sequences)

SEQ ID NO: 1 - DNA, 21 bp, Homo sapiens
atgggatcctaccatggcaag

SEQ ID NO: 2 - DNA, 24 bp, Homo sapiens
gtcttgtttcccagatttcaggaa

SEQ ID NO: 3 - DNA, 22 bp, Homo sapiens
tgggagacagcctcttacttgc

SEQ ID NO: 4 - DNA, 20 bp, Homo sapiens
gcggcggtgagagaaagtct

SEQ ID NO: 5 - DNA, 31 bp, Homo sapiens
agagtggatagcttaaaagaaaaaaagtttc

SEQ ID NO: 6 - DNA, 20 bp, Homo sapiens
cagctcggtccaatcccttc

SEQ ID NO: 7 - DNA, 30 bp, Homo sapiens
acgttggatgcactgaaagagttctacggg

SEQ ID NO: 8 - DNA, 30 bp, Homo sapiens
acgttggatgatgtgaaacccagaacccag

SEQ ID NO: 9 - DNA, 30 bp, Homo sapiens
acgttggatgccacagaaatgcagaatgcc

SEQ ID NO: 10 - DNA, 30 bp, Homo sapiens
acgttggatgcgggcactaatgattcttcc

SEQ ID NO: 11 - DNA, 30 bp, Homo sapiens
acgttggatgtccgggccatagaggacatt

SEQ ID NO: 12 - DNA, 30 bp, Homo sapiens
acgttggatgtgtactctctgcaagggatc

SEQ ID NO: 13 - DNA, 19 bp, Homo sapiens
gggaagatgctaagaaatc

SEQ ID NO: 14 - DNA, 18 bp, Homo sapiens
caggcgcagtgattcagt

SEQ ID NO: 15 - DNA, 17 bp, Homo sapiens
gaatgccactctgaatt

(19) United States
(12) Patent Application Publication
(10) Pub. No.: US 2009/0311692 A1
(43) Pub. Date: Dec. 17, 2009

(54) ISOLATION OF NUCLEIC ACID FROM MOUTH EPITHELIAL CELLS

(76) Inventors: Jerome S. Brody, Newton, MA (US); Avrum Spira, Newton, MA (US)

Correspondence Address: MORSE, BARNES-BROWN & PENDLETON, P.C.;ATTN: IP MANAGER, RESERVOIR PLACE, 1601 TRAPELO ROAD, SUITE 205, WALTHAM, MA, 02451, US

(21) Appl. No.: 12/351,484

(22) Filed: Jan. 9, 2009

Related U.S. Application Data
(63) Continuation of application No. 10/579,376, filed on May 12, 2006, now abandoned.
(60) Provisional application No. 60/519,103, filed on Nov. 12, 2003.
(60) Provisional application No. 60/540,929, filed on Jan. 30, 2004.

(51) Int. Cl. C12Q 1/68

(52) U.S. Cl. 435/6

(57) ABSTRACT

The present invention is directed to a scraping instrument for collection of a biological sample, and a non-invasive method for obtaining nucleic acid from buccal mucosa epithelial cells using the scraping instrument. Such nucleic acid can be used for example for gene expression profiling, including to assess lung disease risk associated with airway pollutants.

### RELATED APPLICATIONS

This application is a continuation of U.S. application Ser. No. 10/579,376, which is a national stage application of PCT/US2004/037764, filed May 12, 2006, which claims the benefit under 35 U.S.C. 119(e) of U.S. Provisional Application Nos. 60/519,103, filed on Nov. 12, 2003, and 60/540,929, filed Jan. 30, 2004, the contents of which are incorporated herein by reference in their entirety. International Application No. PCT/US2004/037764 was published in English.

### GOVERNMENT SUPPORT

This invention was made with Government Support under Contract Number R21-HL71771 awarded by the National Institutes of Health. The Government has certain rights in the invention.

### FIELD OF THE INVENTION

The present invention is directed to a method for isolating nucleic acid from mouth epithelial cells, devices to use for obtaining such nucleic acid, and applications of the nucleic acid obtained.

### BACKGROUND OF THE INVENTION

Substantial interest has been directed to obtaining RNA from various sites and tissues. Increasingly, measurement of gene expression is used as a tool for understanding the pathogenesis of disease and for establishing diagnoses and prognosis of various diseases and disorders, such as cancer, as well as other applications.

The ability to determine gene expression of epithelial cells obtained from the respiratory tract has important implications. For example, the ability to develop an early screening and diagnostic technique for determining whether an individual, who has been exposed to an environmental pollutant such as an irritant or cigarette smoke, has developed or is at risk for developing lung cancer. The epithelial cells of the entire respiratory tract, both intrathoracic and extrathoracic airways, are exposed to environmental pollutants including cigarette smoke and thus can harbor evidence of genetic damage in such individuals. The ability to detect this type of damage may indicate whether individuals have or are at risk for developing lung cancers, and the type thereof.

Lung cancer, environmental pollution, and in particular smoking, remain significant health problems. Smoking is responsible for more than 90% of lung cancer, yet only 15% of smokers actually develop lung cancer. Once it has developed, lung cancer is almost universally fatal, with a 5 year survival rate of only 10-15%. Lung cancer causes more deaths in the United States, approximately 160,000 a year, than the next most common four types of cancer combined. In addition, 25 million current and 25 million former smokers in the U.S. are at risk for developing lung cancer. One of the biggest problems with lung cancer is early detection. In treating cancer, it is well known that early detection of individuals at high risk is extremely important for survival. In dealing with lung cancer, the development of a non-invasive test would be very helpful.

Thus, there is significant interest in developing a simple non-invasive screening tool for assessing an individual's lung cancer risk, including the presence of lung cancer and the risk of developing it in the future, for example by identifying marker genes which have their expression altered at various states of disease progression. Currently, however, such studies use epithelial cells that have been brushed for the large bronchi (intrapulmonary airways) of the lung. Such present processes typically involve bronchoscopy, an invasive procedure with some risk to the patient. It would be desirable to extend the studies to the extrapulmonary airways, using a method to isolate RNA from epithelial cells from the mouth. If one could use RNA obtained from the mouth, it would substantially reduce risk to the subject and samples potentially could be obtained in outpatient or in a large survey setting with ease. However, as discussed below, the environment of the mouth has prevented readily obtaining intact RNA.

Unfortunately, no one has been able to obtain high quality RNA from mouth epithelial cells, also known as buccal mucosa, without invasive biopsy procedures. While swabs and scrapings from the buccal mucosa in the mouth have been used to obtain DNA from epithelial cells for genetic studies^{1,2}, RNA has been obtained from resected tissues and from biopsy samples of mouth epithelium. This is then used in various disease states in order to measure gene expression^{3,4}.

One major barrier to non-invasively obtaining RNA from mouth epithelial cells is saliva, which contains enzymes that degrade RNA (RNAses)⁵. This barrier is further complicated by the fact that scraping cells from the mouth induces salivation and the release of such RNAases. In addition, biopsies of mouth tissue include smooth muscle and other non-epithelial cells. Samples containing such mixed populations of cells are not desirable for all studies. For example, smooth muscle and non-epithelial cells are likely not affected by environmental pollutants such as cigarette smoke.

Accordingly, it would be desirable to have a method and device to obtain intact mouth epithelial cells and extract RNA. Samples of isolated mouth RNA are useful for a wide variety of applications, including studies to measure gene expression.

### SUMMARY OF THE INVENTION

We have developed a novel scraping instrument to collect cells from a subject's mouth, specifically the buccal mucosa epithelial cells, which allows the isolation of nucleic acids, including RNA and DNA. We have also developed a non-invasive method for obtaining nucleic acid from cells in the interior of the mouth, preferably buccal mucosa epithelial cells, using this scraping instrument to collect the epithelial cells. We have also shown that exposure of the mouth to pollutants such as cigarette smoke alters the expression of certain genes in the epithelial cells lining the mouth. The methods of the present invention also provide nucleic acid-based tools to assess lung disease risk associated with exposure to airway pollutants. Nucleic acid tools include analysis of gene expression profiling as well as analysis of DNA methylation patterns.

Accordingly, the invention provides a scraping instrument which has a proximal handle end, a distal collection end, and a joining portion between the handle end and the collection end; wherein the joining portion allows the handle end and the collection end to be optionally detached from each other; and wherein the collection end further comprises a peripheral edge and a depression, wherein at least some of the peripheral edge of said collection portion is serrated to allow scraping of the biological sample, and the depression allows the scraped biological sample to be collected. Preferably, the joining portion is generally continuous in width with the handle end and the collection end on either side of the joining portion.

One preferred scraping instrument has a collection end which is spoon shaped. In yet another embodiment, die scraping instrument is plastic. In another embodiment, the instrument is rubber.

In one preferred embodiment, the joining portion of the scraping instrument comprises a perforation. In another embodiment, the joining portion is not as thick as the handle end and the collection end it is in contact with.

In yet another preferred embodiment, the length of the scraping instrument from about the proximal end of the handle end to the distal end of the collection end is about 3.5-6 inches, and all variants therein. For example 4.0 inches, 4.5 inches, 5.0 inches. In one preferred scraping instrument, the length of the collection end is about 1-2 inches, such as 1.25 inches.

The length and the width of the collection end are designed to permit the collection end to fit into a storage vessel. In one preferred embodiment, the storage vessel contains a lid, which is preferably attached to the storage vessel. Preferably, the storage vessel and the collection end are designed so that the collection end fits snugly in the collection vessel. Typically, some type of solution will also be added to the storage vessel to stably store the biological sample collected.

One embodiment of the present invention provides the non-invasive isolation of a biological sample, wherein the sample is comprised of epithelial cells from buccal mucosa of a subject.

In one preferred embodiment, the scraping instrument of the present invention is used to isolate a biological sample which contains a nucleic acid. Preferably, RNA or DNA. In one embodiment, the nucleic acid is RNA. In another embodiment, the nucleic acid is DNA. Preferably, the nucleic acid such as RNA is from epithelial cells from the buccal mucosa.

One preferred embodiment of the invention provides a non-invasive method to collect a nucleic acid sample from a subject's mouth, involving isolating cells from a subject's mouth using a scraping instrument, transferring the scraped cells to a storage vessel containing a nucleic acid stabilization solution, i.e. one which inhibits the activity of nucleases, and thereafter extracting the nucleic acid from the sample of scraped cells stored in the nucleic acid stabilization solution.

In one embodiment, the sample of scraped cells in the nucleic acid stabilization solution may be stored at −20° C. prior to extraction of the nucleic acid from the sample. In another embodiment, the sample may be shipped to a central lab for analysis.

In one preferred embodiment, the nucleic acid is RNA and the stabilization solution is an aqueous solution that inactivates RNAases and stabilizes RNA, such as “RNA Later” solution (available from Qiagen, Valencia, Calif.).

Any method capable of extracting intact RNA from the sample may be used. One preferred method is the use of TRIzol reagent (available from Invitrogen, Carlsbad, Calif.).

In one preferred embodiment, about 200-2000 ng total RNA is isolated. In another embodiment, about 1000 ng is isolated.

Another preferred embodiment of the invention provides a kit containing a scraping instrument for collecting a biological sample, a storage vessel, and a nucleic acid stabilizing solution.

Yet another preferred embodiment of the present invention provides an RNA collection system, comprising a scraping instrument having a proximal handle end, a distal collection end comprising a serrated peripheral edge, and a joining portion between the handle end and the collection end, wherein the joining portion allows the handle end and the collection end to be optionally detached from each other; and a storage vessel comprising an RNA stabilization solution. Preferably, the storage vessel contains a lid. Even more preferably, the lid is attached to the storage vessel.

The invention also provides a kit for collecting epithelial cells from buccal mucosa, comprising the scraping instrument and a storage vessel comprising an PNA stabilization solution. In one preferred embodiment, the RNA stabilization solution is RNALater.

One preferred embodiment of the present invention provides a method for collecting a sample, comprising the steps of providing a scraping instrument having a proximal handle end, a distal collection end comprising a serrated peripheral edge, and a joining portion between the handle end and the collection end; providing a storage vessel comprising an RNA stabilization solution; scraping the epithelial cells from the buccal mucosa of subject's mouth with the serrated peripheral edge of the collection end, collecting the scraped epithelial cells in the collection end of the scraping instrument; transferring the scraped epithelial cells into the storage vessel; and pivoting the scraping instrument handle to cause the handle end of the instrument to detach from the collection end at the joining portion, such that the storage vessel comprises the RNA storage solution, the scraped sample, and the collection end of the scraping instrument.

The invention also provides a scraping instrument for collecting a nucleic acid sample, comprising a proximal handle end; a distal collection end; and a joining portion between the handle end and the collection end; wherein the joining portion can be continuous in width with the handle end and the collection end on either side of the joining portion and scored, for example by perforations; or less thick than the handle end and collection end on either side; and the joining portion allows the handle end and the collection end to be optionally detached from each other; and wherein the collection end further comprises a peripheral edge and a depression, wherein at least some of the peripheral edge of said collection portion is serrated to allow scraping of the nucleic acid sample, and the depression allows the scraped nucleic acid sample to be collected.

A non-invasive method for obtaining isolated nucleic acid from mouth epithelial cells, comprising: transferring non-invasively isolated cells from a subject's mouth to a nucleic acid stabilization solution that inactivates nucleases, and extracting the nucleic acid of interest from the isolated cells, to obtain an isolated nucleic acid sample. In one preferred embodiment, the nucleic acid is RNA. Preferably, the cells are isolated non-invasively from the mouth by scraping with the scraping instrument of the present invention.

The nucleic acid, preferably RNA, can stably be stored at temperatures for up to and including room temperature, for up to three days, preferably one to two days, with minimal degradation. The lower the temperature, the longer the RNA can be stored. In one preferred embodiment the non-invasive method for obtaining isolated nucleic acid from mouth epithelial cells, the sample of scraped cells in the RNA stabilization solution is stored at −15 to −25° C. prior to extraction of the RNA from the sample. Preferably, the RNA stabilization solution is RNALater RNA stabilization reagent.

We have discovered that gene expression in buccal mucosa epithelial cells can be used as an indicator of the state (or condition) of lung cells. This permits one to identify individuals having or at risk for developing lung disorders.

In one embodiment, the RNA isolated from mouth epithelial cells can be used for gene expression profiling. In another embodiment, the DNA isolated from mouth epithelial cells can be used for identifying changes thereto such as methylation, by DNA methylation analysis.

One embodiment of the invention provides a method to identify smokers who have or are at risk for developing a disorder such as lung cancer, by profiling buccal epithelial cells for the expression of gene(s) associated with different disorders such as the stages of lung cancer.

Accordingly, one embodiment of the invention provides a method for detecting the expression of a target gene(s) of interest in a sample of buccal mucosa epithelial cells, comprising: isolating a nucleic acid sample from buccal mucosa epithelial cells, as described; contacting the isolated nucleic acid sample of step (a) with at least one nucleic acid probe which specifically hybridizes to the target gene(s) of interest; and detecting the presence of said target gene(s) of interest in the nucleic acid sample. In one embodiment, the target gene(s) of interest is attached to a solid phase prior to performing step (b). Preferably the nucleic acid is RNA or DNA.

In one preferred embodiment, the gene(s) of interest is differentially expressed in subjects who have lung cancer as opposed to subjects not having lung cancer. For example, the gene(s) of interest is expressed in subjects who have lung cancer and not expressed in subjects who do not have lung cancer. Preferably, one looks at least 2 genes, more preferably at least 5 genes of interest.

We have previously found that about 208 genes are differentially expressed in the airway in smokers who have lung cancer as opposed to smokers who do not have lung cancer, which comprise a lung cancer diagnostic airway transcriptome. Similarly, the methods of the present invention also provide methods for identifying differentially expressed genes which comprise a lung cancer diagnostic mouth transcriptome, the expression pattern of which is useful in prognostic, diagnostic and therapeutic applications as described herein. The genes comprising the diagnostic mouth transcriptome are expressed in mouth epithelial cells, and have expression patterns that differ significantly between individuals with lung cancer and healthy individuals. The lung cancer diagnostic mouth transcriptome is also referred to as a smoker's differential mouth transcriptome. The expression patterns of such a lung cancer diagnostic mouth transcriptome are useful in prognosis of lung disease, diagnosis of lung disease and a periodic screening of the same individual to see if that individual has been exposed to risky airway pollutants such as cigarette smoke that change his/her expression pattern.

One embodiment of the invention provides identifying genes which comprise different mouth transcriptomes. One useful mouth transcriptome is comprised of genes which are also expressed in the bronchi and whose expression in the bronchi is differentially affected by a pollutant such as cigarette smoke, and are also expressed in the mouth. Another useful transcriptome is a lung cancer diagnostic mouth transcriptome. One method for identifying the genes which comprises a lung cancer diagnostic mouth transcriptome is to first identify a mouth transcriptome (as described above), and then determining which of those genes are differentially expressed in the mouth of individuals with lung cancer and healthy individuals.

In one embodiment, we have now identified about 166 genes which comprise a mouth transcriptome, i.e. genes which are expressed in the bronchi and whose expression in the bronchi is affected by cigarette smoke, and which are also expressed in the mouth, consisting of the following genes: ABCC1; ABHD2; AF333388.1; AGTPBP1; AIP1; AKR1B10AKR1C1; AKRR1C2; AL117536.1; AL353759; ALDH3A1; ANXA3; APLP2; ARHE; ARL1; ARPC3; ASM3A; B4GALT5; BECN1; C1orf8; C20orf111; C5orf6; C6orf80; CA12; CABYR; CANX; CAP1; CCNG2; CEACAM5; CEACAM6; CED-6; CHP; CHST4; CKB; CLDN10; CNK1; COPB2; COX5A; CPNE3; CRYM; CSTA; CTGF; CYP1B1; CYP2A6; CYP4F3; DEFB1; DIAPH2; DKFZP434J214; DKFZP564K0822; DKFZP566E144; DSCR5; DSG2; EPAS1; EPOR; FKBP1A; FLJ10134; FLJ13052; FLJ130521; FLJ20359; FMO2; FTH1; GALNT1; GALNT3; GALNT7; GCLC; GCLM; GGA1; GHITM; GMDS; GNE; GPX2; GRJP58; GSN; GSTM3; GSTM5; GUK1; HIG1; HIST1H2BK; HN1; HPGD; HRIHFB2122; HSPA2; IDH1; IDS; IMPA2; ITM2A; JTB; KATNB1; KDELR3; KIAA0397; KIAA0905; KLF4; KRT14; KRT15; LAMP2; LOC51186; LOC57228; LOC92482; LOC92689; LYPLA1; MAFG; ME1; MGC4342; MGLL; MT1E; MT1F; MT1G; MT1H; MT1X; MT2A; NCOR2; NKX3-1; NQO1; NUDT4; ORL1; P4HB; PEX14; PGD; PRDX1; PRDX4; PSMB5; PSMD14; PTP4A1; PTS; RAB11A; RAB2; RAB7; RAP1GA1; RNP24; RPN2; S100A10; S100A14; S100P; SCP2; SDR1; SHARP1; SLC17A5; SLC35A3; SORD; SPINT2; SQSTM1; SRPUL; SSR4; TACSTD2; TALDO1; TARS; TCF7L1; TIAM1; TJP2; TLE1; TM4SF1; TM4SF13; TMP21; TNFSFl3; TNS; TRA1; TRIM16; TXN; TXNDC5; TXNL; TXNRD1; UBE2J1; UFD1L; UGT1A10; YF13H12; and ZNF463. The symbols represent the HUGO identification symbols. FIG. 11 lists details of each of the transcripts corresponding to these genes, including the expression ratio of these genes as compared between smokers and non-smokers (current smoker/never smoker ratio) and the p-value, which shows the significance of the difference in expression of these genes in smokers and non-smokers (current smoker/never smoker p-value). FIG. 11 also shows the gene various gene symbols that these genes appear in databases including HUGO, GenBank and GO databases. Also the Affymetrix cDNA chip location of these transcripts is shown. In one embodiment, the expression of these genes between individuals with lung cancer and healthy individuals is compared, in order to identify genes which form a lung cancer diagnostic mouth transcriptome.

In one preferred embodiment, another mouth transcriptome consists of the following genes, identified using their Human Genome Organization (HUGO) identification symbols: AGTPBP1; AKR1C1; AKR1C2; ALDH3A1; ANXA3; CA12; CEACAM6; CLDNl0; CYP1B1; DPYSL3; FLJ13052; FTH1; GALNT3; GALNT7; GCLC; GCLM; GMDS; GPX2; HN1; HSPA2; MAFG; ME1; MGLL; MMP10; MT1F; MT1G; MT1X; NQO1; NUDT4; PGD; PRDX1; PRDX4; RAB11A; S100A10; SDR1; SRPUL; TALDO1; TARS; TCF-3; TRA1; TRIM16; TXN; and TXNRD1. FIG. 12 lists details of each of the identified transcripts corresponding to these genes including the expression ratio of these genes as compared between smokers and non-smokers (smoker/non-smoker expression ratio) and the p-value, which shows the significance of the difference in expression of these genes in smokers and nonsmokers (smoker/non-smoker p-value). In one preferred embodiment, the expression of these genes between individuals with lung cancer and healthy individuals is compared, in order to identify genes which form a lung cancer diagnostic mouth transcriptome. This lung cancer diagnostic mouth transcriptome can then be used to screen for individuals having lung cancer or at risk for developing lung cancer.

One embodiment of the invention provides a method of determining whether an individual is at increased risk of developing a lung disease, comprising: taking a biological sample from the mouth of an individual exposed to an airway pollutant or at risk of being exposed to an airway pollutant; and analyzing whether there is a genetic alteration in at least one gene, preferably two genes, preferably 5-10 genes, preferably 10-100 genes, of the mouth transcriptome genes, wherein the presence of a genetic alteration in one or more of the mouth transcriptome genes as compared to the same at least one gene in a group of control individual is indicative that the individual has an increased risk of developing a lung disease. In one embodiment, the genetic alteration is a deviation of a gene's DNA methylation pattern or a deviation of a gene's expression pattern. In one preferred embodiment, the air pollutant is smoke from a cigarette or a cigar and the lung disease is lung cancer. Preferably, the lung cancer is adenocarcinoma, squamous cell carcinoma, small cell carcinoma, large cell carcinoma, or benign neoplasms of the lung.

In one preferred embodiment, the individual is a smoker and one looks at expression of at least one gene selected from the group consisting of the lung cancer diagnostic mouth transcriptome genes, wherein lower expression of that at least one gene in the smoker than in a control group of corresponding smokers is indicative of an increased risk of developing lung cancer. In another preferred embodiment, one looks at expression of at least three genes of the mouth transcriptome. More preferably, one looks at expression of at least five genes.

In one preferred embodiment, the individual is a smoker and one looks at expression of at least one gene selected from the group consisting of the diagnostic lung cancer mouth transcriptome genes, wherein higher expression of that at least one gene in the smoker than in a control group of corresponding smokers is indicative of an increased risk of developing lung cancer. In another preferred embodiment, one looks at expression of at least three genes of the diagnostic lung cancer mouth transcriptome. More preferably, one looks at expression of at least five genes.

In one preferred embodiment, one looks at genes encoding the expression of aldehyde dehydrogenase (ALDH3A1), NADPH (NQ01), and CEACAM5 (CEACAM5).

In yet another preferred embodiment, the individual is a smoker and one looks at expression of at least one gene selected from the diagnostic lung cancer mouth transcriptomes encoding proto-oncogenes, wherein higher or lower expression of that at least one gene in the smoker than in a control group of corresponding smokers is indicative of an increased risk of developing lung cancer. In one preferred embodiment, higher or lower expression of at least one gene in each of the mouth transcriptome encoding proto-oncogenes is indicative of an increased risk of developing lung cancer.

In yet another preferred embodiment, the individual is a smoker and one looks at expression of at least one gene selected from the diagnostic lung cancer mouth transcriptomes encoding a tumor suppressor gene, wherein higher or lower expression of that at least one gene in the smoker than in a control group of corresponding smokers is indicative of an increased risk of developing lung cancer. ID one embodiment, higher or lower expression of at least one gene in each of the diagnostic lung cancer mouth transcriptome encoding a tumor suppressor gene is indicative of an increased risk of developing lung cancer.

The present invention also provides a method of diagnosing the predisposition of a smoker or a non-smoker to lung disease comprising analyzing an expression pattern of one or more genes selected from the group consisting of ABCC1; ABHD2; AF333388.1; AGTPBP1; A1P1; AKR1B10AKR1C1; AKR1C2; AL117536.1; AL353759; ALDH3A1; ANXA3; APLP2; ARHE; ARL1; ARPC3; ASM3A; B4GALT5; BECN1; C1orf8; C20orf111; C5orf6; C6orf80; CA12; CABYR; CANX; CAP1; CCNG2; CEACAM5; CEACAM6; CED-6; CHP; CHST4; CKB; CLDN10; CNK1; COPB2; COX5A; CPNE3; CRYM; CSTA; CTGF; CYP1B1; CYP2A6; CYP4F3; DEFB1; DIAPH2; DKFZP434J214; DKFZP564K0822; DKFZP566E144; DSCR5; DSG2, EPAS1; EPOR; FKBP1A; FLJ10134; FLJ13052; FLJ130521; FLJ20359; FMO2; FTH1; GALNT1; GALNT3; GALNT7; GCLC; GCLM; GGA1; GHITM; GMDS; GNE; GPX2; GRP58; GSN; GSTM3; GSTM5; GUK1; HIG1; HIST1H2BK; HN1; HPGD; HRIHFB2122; HSPA2; IDH1; IDS; IMPA2; ITM2A; JTB; KATNB1; KDELR3; KIAA0397; KIAA0905; KLF4; KRT14; KRT15; LAMP2; LOC51186; LOC57228; LOC92482; LOC92689; LYPLA1; MAFG; ME1; MGC4342; MGLL; MT1E; MT1F; MT1G; MT1H; MT1X; MT2A; NCOR9; NKX3-1; NQO1; NUDT4; ORL1; P4HB; PEX14; PGD; PRDX1; PRDX4; PSMB5; PSMD14; PTP4A1; PTS; RAB11A; RAB2; RAB7; RAP1GA1; RNP24; RPN2; S100A10; S100A14; S100P; SCP2; SDR1; SHARP1; SLC17A5; SLC35A3; SORD; SPINT2; SQSTM1; SRPUL; SSR4; TACSTD2; TALDO1; TARS; TCF7L1; TIAM1; TJP2; TLE1; TM4SF1; TM4SF13; TMP21; TNFSF13; TNS; TRA1; TRIM16; TXN; TXNDC5; TXNL; TXNRD1; UBE2J1; UFD1L; UGT1A10; YF13H12; and ZNF463. In one preferred embodiment, the expression pattern of one or more genes selected from the group consisting of: AGTPBP1; AKR1C1; AKR1C2; ALDH3A1; ANXA3; CA12; CEACAM6; CLDN10; CYP1B1; DPYSL3; FLJ13052; FTH1; GALNT3; GALNT7; GCLC; GCLM; GMDS; GPX2; HN1; HSPA2; MAFG; ME1; MGLL; MMP10; MT1F; MT1G; MT1X; NQO1; NUDT4; PGD; PRDX1; PRDX4; RAB11A; S100A10; SDR1; SRPUL; TALDO1; TARS; TCF-3; TRA1; TRIM16; and TXN. Preferably, the expression pattern of one or more genes is analyzed in a biological sample taken from the mouth of the smoker or the non-smoker, wherein a divergent expression pattern of one or more of these genes as compared to the expression pattern of these genes in group of control individuals is indicative of the predisposition of the individual to lung disease. In one preferred embodiment the lung disease is lung cancer, including adenocarcinoma, squamous cell carcinoma, small cell carcinoma, large cell carcinoma, and benign neoplasms of the lung.

In one embodiment, the present invention provides method for screening for a subject's predisposition to lung disease, wherein the biological sample for diagnosis is a nucleic acid sample. In one preferred embodiment, wherein the nucleic acid is RNA or DNA. Preferably, the sample is RNA. In another preferred embodiment, the analysis is performed using a nucleic acid array. In another preferred embodiment, the analysis is performed using quantitative real time PCR or mass spectrometry.

### BRIEF DESCRIPTION OF THE DRAWINGS

FIG. 1 is a drawing of one embodiment of the invention including an intact scraping instrument with a detachable handle and a serrated collection end, and a storage vessel.

FIG. 2 illustrates an embodiment of the invention showing the collection portion containing the scraped biological sample detached from the handle of the scraping instrument, and a storage vessel containing a nucleic acid stabilization solution.

FIG. 3 illustrates an embodiment of the invention including the detached scraping instrument, with the handle separated from the collection end at the joining portion, and the collection end placed into the storage vessel containing a nucleic acid stabilization solution.

FIG. 4 illustrates an alternative embodiment of the invention with one serrated edge of the collection end of the scraping instrument.

FIG. 5 illustrates several alternative embodiments of the invention, including different shapes for the collection end.

FIG. 6 shows RNA extracted from an epithelial cell line (lane 1) and buccal mucosa scraping (lane 2) on a 1% agarose RNA denaturing gel. Bands for 28s rRNA (upper arrow) and 18s rRNA (lower arrow) are shown. This gel is one of the best examples obtained. Most scrapings produce too little RNA for a gel or displayed evidence for some RNA degradation. This partial degradation did not impair the ability to measure RNA by real time PCR or mass spectrometry.

FIG. 7 shows the results of an immunocytochemical stain for the pancytokeratin protein in buccal mucosa cells obtained using the method of the present invention. All cells have epithelial morphology and stain positive (brown) for the antibody to various degrees.

FIGS. 8A-B show the expression levels for select buccal mucosa epithelial cell genes in smokers and nonsmokers. In FIG. 8A, buccal mucosa epithelial gene expression was measured by real time QRT-PCR. Mean (+/−SD) expression fold changes for 3 never smokers and 2 current smokers for each gene are shown (only one current smoker sample was measured for NQO1). Fold change refers to the ratio of the mean expression level of a gene in a group of samples as compared to one of the non-smoker samples. All real time PCR experiments were carried out in duplicate on each sample. In FIG. 8B, buccal mucosa epithelial gene expression was measured by competitive PCR and MALDI TOF mass spectrometry. Expression levels were normalized to total RNA concentration (10⁻⁷ μM/μg total RNA). Mean (+/−SD) expression level for 7 never smokers and 10 current smokers for each gene are shown. There was a significant (p<0.05) increase in gene expression for ALDH3A1 and NQO1 in current smokers.

FIG. 9 shows the correlation of the expression of several genes in the airway and the mouth. The data show the fold-change of three genes, ALDH3A1, CEACAM5, and NQO1, in people who have never smoked (“Never smokers”) and current smokers. In addition, two gene expression detection techniques are compared here: mass spectroscopy and gene arrays.

FIG. 10 illustrates three major problems presented by lung cancer. While 85% of lung cancer is found in current or former smokers, only 15% of smokers develop lung cancer. A first issue is identifying those individuals who have a susceptibility to develop lung cancer, which is critical to both early diagnosis and prognosis. 15% of lung cancers are diagnoses when the cancer is still highly localized; for these patients, 5 year survival is 50%. However, for the 50% of lung cancer patients diagnosed with distal cancer, 5 year survival is less than 5%. Thus, early diagnosis is critical.

FIG. 11 shows a list of genes the expression of which is affected by cigarette smoke in bronchi. These genes are also expressed in mouth epithelial cells.

FIG. 12 shows a subset of genes listed in FIG. 11, the expression of which is most affected by cigarette smoke in bronchi. These genes are also expressed in mouth epithelial cells.

### DETAILED DESCRIPTION OF THE INVENTION

We have now discovered a non-invasive method for obtaining nucleic acid from cells in the interior of the mouth. We have also invented a scraping instrument for collection of a biological sample, and a non-invasive method for obtaining nucleic acid from buccal mucosa epithelial cells using the scraping instrument. The methods of the present invention also provide nucleic acid-based tools to assess lung disease risk associated with exposure to airway pollutants. Nucleic acid tools include analysis of gene expression profiling as well as analysis of DNA methylation patterns.

We have also shown that exposure of the mouth to pollutants such as cigarette smoke alters the expression of certain genes in the epithelial cells lining the mouth. For example, lung cancer involves histopathological and molecular progression from normal to premalignant to cancer. Gene expression arrays of lung tumors have been used to characterize expression profiles of lung cancers, and to show the progression of molecular changes from non-malignant lung tissue to lung cancer. However, for the screening and early diagnostic purpose, it is not practicable to obtain samples from the lungs. Therefore, the present invention provides for the first time, a method of obtaining cells from the mouth, the most accessible part of the airway, to identify the epithelial gene expression pattern in an individual.

The ability to determine which individuals have molecular changes in their airway epithelial cells and how these changes relate to a lung disorder, such as premalignant and malignant changes, is a significant improvement for determining risk and for diagnosing a lung disorder such as cancer at a stage when treatment can be more effective, thus reducing the mortality and morbidity rates of lung cancer. The case with which the present invention allows airway epithelial cells to be obtained from buccal mucosal scrapings shows that this approach has wide clinical applicability and is a useful tool in a standard clinical screening for the large number of subjects at risk for developing disorders of the lung.

In one embodiment, the RNA isolated from mouth epithelial cells can be used for gene expression profiling. In another embodiment, the DNA isolated from mouth epithelial cells can be used for DNA methylation analysis.

One embodiment of the invention provides a method to identify smokers who have or are at risk for developing lung cancer, by profiling buccal epithelial cells for the expression of gene(s) associated with different stages of lung cancer.

### Scraping Instrument

The scraping instrument permits one to non-invasively collect cells from a subject's mouth which allows the isolation of nucleic acids, including RNA and DNA. The tool has two features that allow collection of a significant amount of good quality nucleic acid, including RNA, from the buccal mucosa: a finely serrated edge that can scrape off several layers of epithelial cells, and a concave surface (or depression) in the collection end to collect the scraped cells.

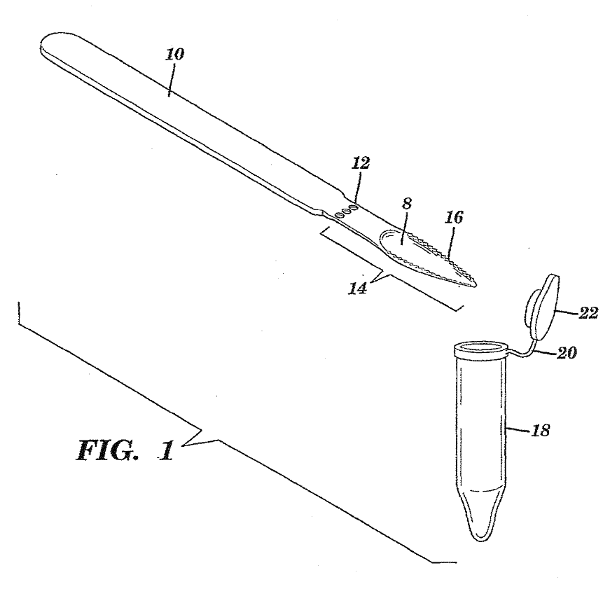

Referring to the figures where like reference numerals indicate like elements, FIG. 1 illustrates an exemplary embodiment of the invention, including an intact scraping instrument with a handle and a serrated collection end, and a storage vessel. The scraping instrument has a proximal handle end 10, a distal collection end 14, and a joining portion 12 between the handle end 10 and the collection end 14; wherein the joining portion 12 is generally continuous in width with the handle end 10 and the collection end 14 on either side of the joining portion 12. The joining portion 12 allows the handle end 10 and the collection end 14 to be optionally detached from each other. The collection end 14 further comprises a peripheral edge 16 and a depression 8, wherein at least some of the peripheral edge 16 is serrated to allow scraping of the biological sample, and the depression 8 allows the scraped biological sample to be collected. The storage vessel 18 in this embodiment has a lid 22 attached to the storage vessel 18 by a connector 20.

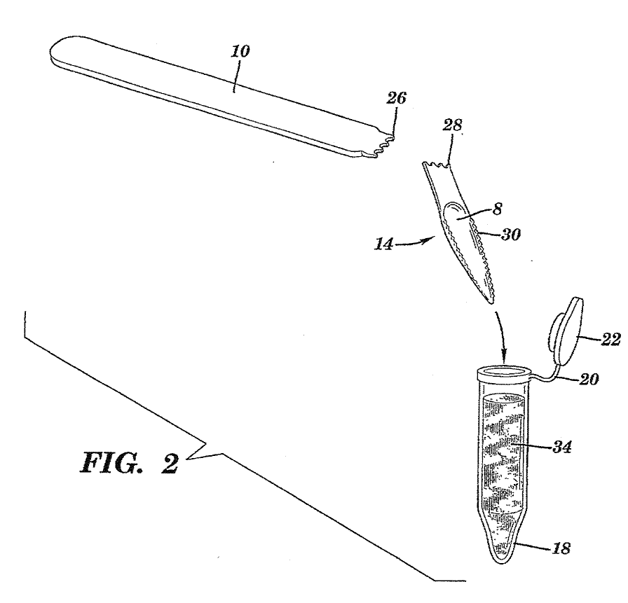

FIG. 2 illustrates an embodiment of the invention as illustrated in FIG. 1, wherein the handle end 10 has been detached from the collection end 14. The detachment comes by the joining end being scored by perforations that detach at ends 26 and 28. The storage vessel 18 contains a nucleic acid stabilization solution 34.

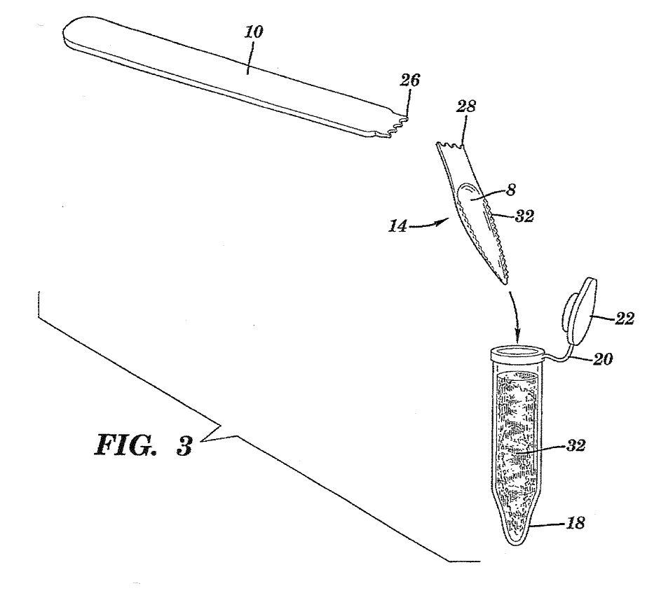

FIG. 3 illustrates the embodiment of the invention illustrated in FIGS. 1 and 2, where the scraping instrument is detached, with the handle separated from the collection end at the joining portion, and the collection end placed into the storage vessel containing a nucleic acid stabilization solution. The handle end 10 is detached from the collection end 14. The collection end 14 of the scraping instrument is placed in the storage vessel 18 which contains the nucleic acid stabilization solution 34 and contains a biological sample 32. In this embodiment, the storage vessel 18 also has a lid 22 and a connector 20 which joins the lid 22 to the storage vessel 18.

One preferred embodiment provides a plastic or some other polymeric tool, as illustrated in FIGS. 1-3, that has a serrated edge to scrape off several layers of epithelial cells, and a curved surface to collect those cells. In this embodiment, a standardized plastic tool that has a spoon-shaped end which is concave with serrated edges, for example 5/16 inches wide and 1 6/16 inches long, with a 3 inch handle that can be broken off when the scraping tool with collected cells is inserted into a storage vessel, such as a 2 ml microfuge tube.

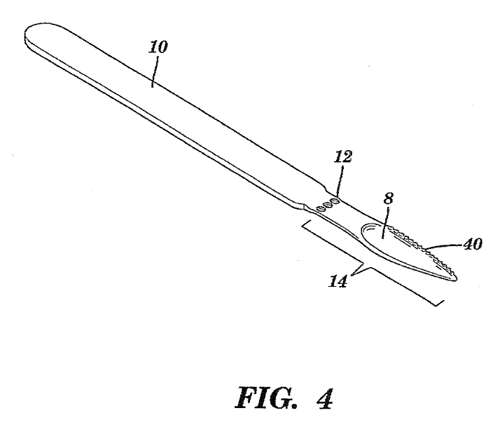

Any portion of the peripheral edge of the collection end can be serrated. In one embodiment, as depicted in FIGS. 1-3, the entire peripheral edge of the collection end is serrated. However, the invention comprises other embodiments in which less than the entire peripheral edge is serrated. For example, FIG. 4 illustrates an alternative embodiment of the invention with one side serrated, that is 50%, of the peripheral edge 40 of the collection end 14 of the scraping instrument.

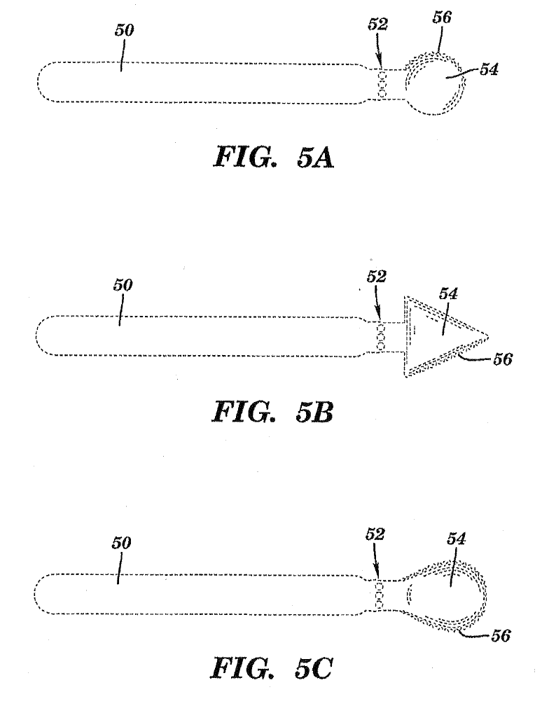

The collection end of the scraping instrument can have any shape. One preferred scraping instrument has a collection end which is spoon shaped. FIG. 5 illustrates several embodiments, all of which have a handle end 50 connected to a collection end 54 by a joining portion 52, where the collection end has a serrated peripheral edge 56.

The scraping instrument of the present invention can be made of any material which allows the handle end and the collection end to be detachable connected via a joining portion. In one preferred embodiment, the scraping instrument is plastic.

The joining portion can have any design or construction which allows the handle end and the collection end to be optionally detached. In one preferred embodiment, the joining portion of the scraping instrument comprises a perforation. In this embodiment, when the handle end of the instrument is pivoted back and forth, the collection end detaches from the handle at the site of the perforation. In another embodiment, the joining portion is thinner than the adjoining handle end and collection end.

The scraping instrument can be any size which allows its functioning in the collection of a sample. In one preferred embodiment, the length of the scraping instrument from about the proximal end of the handle end to the distal end of the collection end is about 3.5 to 6 inches and all variants therein, for example 4.5 inches. In one preferred scraping instrument, the length of the collection end is about 1-2 inches and all variants therein, such as 1.25 inches.

The length and the width of the collection end of the instrument are designed to allow the collection end to fit into a storage vessel. In one preferred embodiment, the storage vessel contains a lid, which is preferably attached to the storage vessel.

In another embodiment, the scraping instrument is a pipette tip that has been cut in half to generate a curved surface for scraping the surface of the mouth to collect cells.

The scraping instrument of the present invention can be used for the isolation and collection of any sample of interest. In one preferred embodiment, the sample is a biological sample. In a particularly preferred embodiment, the sample is a large number of epithelial cells from the buccal mucosa.

### Collection and Storage of Nucleic Acid Sample

The invention provides a non-invasive method to collect a nucleic acid sample from a subject's mouth, involving isolating cells from a subject's mouth using the scraping instrument, transferring the scraped cells to a storage vessel containing a nucleic acid stabilization solution, i.e. one which inhibits the activity of nucleases, and extracting the nucleic acid from the sample of scraped cells in the nucleic acid stabilization solution. Thereafter, the sample is stored until analyzed.

To collect a sample from a subject's mouth, the scraping instrument is used. Using gentle pressure, the serrated edge can be scraped, for example four-ten times, against the buccal mucosa on the inside of the cheek, and the collected cells can be immediately immersed in an nucleic acid stabilization solution, for example by placing the collection end of the instrument into a storage vessel.

In one preferred embodiment, the scraping instrument of the present invention is used to isolate a biological sample which contains a nucleic acid. Preferably, RNA or DNA. In one embodiment, the nucleic acid is RNA. In another embodiment, the nucleic acid is DNA. The stored sample can then be sent for analysis.

In one embodiment, the sample of scraped cells in the nucleic acid stabilization solution may be stored at any temperature from up to and including room temperature (about 22° C.) to −30° C. The lower the temperature the longer the sample can stably be stored. Preferably, the temperature is −5° C. to −30° C., more preferably −15° C. to −20° C., still more preferably −20° C. prior to extraction of the nucleic acid from the sample. In another embodiment, the sample may be stored at 4° C. for 24-96 hours prior to extraction of the nucleic acid from the sample. Even more preferably, 24 hours.

In a particularly preferred embodiment, the sample of scraped cells in the nucleic acid stabilization solution may be stored at room temperature for 24 to 72 hours prior to extraction of the nucleic acid from the sample. The sample can thus be sent from the site of extraction to a central location for analysis.

The sample of scraped cells of the present invention can be transferred into any storage vessel suitable for storage of the nucleic acid contained within the sample. Such vessels are well known in the art and available from many sources. In one preferred embodiment, the storage vessel is a small tube, such as a microfuge tube, which readily allows further processing of the sample. For example, a plastic tube with a volume of approximately 1.5-2 milliliters. In one preferred embodiment, the storage vessel has the size and shape to accommodate the collection end of the scraping instrument once it has been detached from its handle end. Even more preferably, the storage vessel has a lid, and the lid can be closed after the collection end of the scraping instrument has been placed into the vessel. Preferably the lid of the storage vessel is attached to the vessel.

The storage vessel preferably contains a solution suitable for the transfer and storage of the sample, to allow preservation of the nucleic acid of interest. Preferably, the stabilization solution inactivates any nucleases which degrade the nucleic acid of interest. If the nucleic acid is RNA, the stabilization solution inactivates RNAses. If the nucleic acid is DNA, the stabilization solution inactivates DNAses.

In one preferred embodiment, the nucleic acid is RNA and the stabilization solution inactivates at least 75% of RNAase activity within 5 minutes, preferably it inactivates at least 75% of RNAase activity within one minute. Still more preferably, it inactivates at least 85% of RNAase activity within 4 minutes of submersion of the RNA. Even more preferably, it inactivates at least 85% of RNAase activity within one minute of submersion of the RNA. Yet more preferably, it inactivates at least 90% of RNAase activity within two minutes of submersion of RNA, still more preferably at least 90% of RNAase activity within one minute of submersion of RNA. Still more preferably it inactivates at least 95% of RNAase activity within two minutes of submersion. Even more preferably it inactivates at least 95% of RNAase activity within one minute of submersion.

Any RNA stabilization solution that allows the recovery of intact total RNA may be used to store the collected sample. In one preferred embodiment, the RNA stabilization solution is “RNALater” stabilization reagent available from Qiagen, Valencia, Calif.

In one preferred embodiment, the method of the present invention can be used to isolate large quantities of isolated buccal epithelial cell RNA. Preferably, a single isolation procedure generates nanogram-microgram quantities of RNA. In one preferred embodiment, about 200-2000 ng total RNA is isolated. In one preferred embodiment, about 1000 ng is isolated.

The isolated buccal epithelial cell RNA of the present invention can be used in any method or procedure for which it is desirable to have such total intact RNA.

Nucleic acids that are obtained from a buccal epithelial cell sample can be isolated by any standard means known to a skilled artisan. Standard methods of DNA and RNA isolation, as well as recombinant nucleic acid methods used herein generally, are described in Sambrook et al., Molecular Biology. A laboratory Approach, Cold Spring Harbor, N.Y. 1989; Ausubel, et al., Current protocols in Molecular Biology, Greene Publishing, Y, 1995.

The nucleic acid of interest can be recovered or extracted from the stabilization solution by any suitable technique that results in isolation of the nucleic acid from at least one component of the stabilization solution. Using known means one can also identify what cells the nucleic acid is coming from. Nucleic acid can be recovered from the stabilization solution by extraction with an organic solvent, chloroform extraction, phenol-chloroform extraction, precipitation with ethanol, isopropanol or any other lower alcohol, by chromatography including ion exchange chromatography, size exclusion chromatography, silica gel chromatography and reversed phase chromatography, or by electrophoretic methods, including polyacrylamide gel electrophoresis and agarose gel electrophoresis, as will be apparent to one of skill in the art. Nucleic acid is preferably recovered from the stabilization solution using phenol chloroform extraction.

One particularly preferred method for extracting intact RNA from the sample is the use of TRIzol reagent (available from Invitrogen, Carlsbad, Calif.).

Following nucleic acid recovery, the nucleic acid may optionally be further purified by techniques which are well known in the art. In one preferred embodiment, further purification results in RNA that is substantially free from contaminating DNA or proteins. Further purification may be accomplished by any of the aforementioned techniques for nucleic acid recovery. Nucleic acid is preferably purified by precipitation using a lower alcohol, especially with ethanol or with isopropanol. Precipitation is preferably carried out in the presence of a carrier such as glycogen that facilitates precipitation.

The nucleic acid samples of the present invention may be amplified by a variety of mechanisms, some of which may employ PCR. See, e.g., PCR Technology: Principles and Applications for DNA Amplification (Ed. H. A. Erlich, Freeman Press, NY, N.Y., 1992); PCR Protocols: A Guide to Methods and Applications (Eds. Innis, et al., Academic Press, San Diego, Calif., 1990); Mattila et al., Nucleic Acids Res. 19, 4967 (1991); Eckert et al., PCR Methods and Applications 1, 17 (1991); PCR (Eds. McPherson et al., IRL Press, Oxford); and U.S. Pat. Nos. 4,683,202, 4,683,195, 4,800,159 4,965,188, and 5,333,675, each of which is incorporated herein by reference in their entireties for all purposes. The sample may be amplified on the array. See, for example, U.S. Pat. No. 6,300,070 and U.S. patent application Ser. No. 09/513,300, which are incorporated herein by reference.

Other suitable amplification methods include the ligase chain reaction (LCR) (e.g., Wu and Wallace, Genomics 4, 560 (1989), Landegren et al., Science 241, 1077 (1988) and Barringer et al, Gene 89:117 (1990)), transcription amplification (Kwoh et al., Proc. Natl. Acad. Sci. USA 86, 1173 (1989) and WO88/10315), self-sustained sequence replication (Guatelli et al., Proc. Nat. Acad. Sci. USA, 87, 1874 (1990) and WO90/06995), selective amplification of target polynucleotide sequences (U.S. Pat. No. 6,410,276), consensus sequence primed polymerase chain reaction (CP-PCR) (U.S. Pat. No. 4,437,975), arbitrarily primed polymerase chain reaction (AP-PCR) (U.S. Pat. Nos. 5,413,909, 5,861,245) and nucleic acid based sequence amplification (NABSA). (See, U.S. Pat. Nos. 5,409,818, 5,554,517, and 6,063,603, each of which is incorporated herein by reference). Other amplification methods that may be used are described in, U.S. Pat. Nos. 5,242,794, 5,494,810, 4,988,617 and in U.S. Ser. No. 09/854,317, each of which is incorporated herein by reference.

RNA isolated by the method of the present invention can include messenger RNA (mRNA), transfer RNA (tRNA), ribosomal RNA (rRNA), and viral RNA.

RNA isolated by the methods of the present invention is suitable for a variety of purposes and molecular biology procedures including, but not limited to: reverse transcription to cDNA; producing radioactively, fluorescently or otherwise labeled cDNA for analysis on gene chips, oligonucleotide microarrays and the like; electrophoresis by acrylamide or agarose gel electrophoresis; purification by chromatography (e.g. ion exchange, silica gel, reversed phase, or size exclusion chromatography); hybridization with nucleic acid probes; and fragmentation by mechanical, sonic or other means. Common methods for analyzing RNA include northern blotting, ribonuclease protection assays (RPAs), reverse transcriptase-polymerase chain reaction (RT-PCR), quantitative real-time PCR, cDNA preparation for cloning, in vitro translation and microarray analyses.

DNA isolated by methods of the present invention is suitable for a variety of purposes and molecular biology procedures including, but not limited to: producing radioactively, fluorescently or otherwise labeled DNA for analysis on gene chips, oligonucleotide microarrays and the like; electrophoresis by acrylamide or agarose gel electrophoresis; purification by chromatography (e.g. ion exchange, silica gel, reversed phase, or size exclusion chromatography); hybridization with nucleic acid probes; and fragmentation by mechanical, sonic or other means. Common methods for analyzing DNA include Southern blotting, polymerase chain reaction (PCR), quantitative real-time PCR, cloning, in vitro transcription and translation, and microarray analyses.

One preferred embodiment of the invention provides a kit containing a scraping instrument for collecting a biological sample, a storage vessel, and a nucleic acid stabilizing solution.

Yet another preferred embodiment of the present invention provides an RNA collection system, comprising a scraping instrument having a proximal handle end, a distal collection end comprising a serrated peripheral edge, and a joining portion between the handle end and the collection end, where the joining portion allows the handle end and the collection end to be optionally detached from each other; and a storage vessel comprising an RNA stabilization solution. Preferably, the storage vessel contains a lid. Even more preferably, the lid is attached to the storage vessel.

The invention also provides a kit for collecting epithelial cells from buccal mucosa, comprising the scraping instrument and a storage vessel comprising an RNA stabilization solution. In one preferred embodiment, the RNA stabilization solution is RNALater.

One preferred embodiment of the present invention provides a method for collecting a sample, comprising the steps of providing a scraping instrument having a proximal handle end, a distal collection end comprising a serrated peripheral edge, and a joining portion between the handle end and the collection end; providing a storage vessel comprising an RNA stabilization solution; scraping the epithelial cells from the buccal mucosa of subject's mouth with the serrated peripheral edge of the collection end; collecting the scraped epithelial cells in the collection end of the scraping instrument; transferring the scraped epithelial cells into the storage vessel; and pivoting the scraping instrument handle to cause the handle end of the instrument to detach from the collection end at the joining portion, such that the storage vessel comprises the RNA storage solution, the scraped sample, and the collection end of the scraping instrument.

As discussed below, the nucleic acids isolated from these mouth epithelial cells are indicative of the conditions of lung cells. This permits the creation of non-invasive tests involving the lung.

### Lung Disorder Biomarkers

We have also discovered that gene expression in buccal mucosa epithelial cells can be used as an indicator of the state (or condition) of lung cells. This permits one to identify individuals having or at risk for developing lung disorders, such as lung cancer.

We have shown that exposure of airways, including the mouth, to pollutants such as cigarette smoke, causes a so-called “field defect”, which refers to gene expression changes in all the epithelial cells lining the airways from mouth mucosal epithelial lining through the bronchial epithelial cell lining to the lungs (Spira et al., Proc Natl. Acad. Sci. USA. 2004 Jul. 6; 101(27):10143-8). See also International Application PCT/US04/18460. Because of this field defect, it is now possible to detect changes, for example, pre-malignant and malignant changes resulting in diseases of the lung, using cell samples isolated from epithelial cells obtained not only from the lung biopsies but also from other, more accessible, parts of the airways including mouth epithelial cell samples.

One aspect of the present invention is based on the finding that that there are different patterns of gene expression between smokers and non-smokers (Spira et al., 2004). Another aspect of the invention is based on the finding that another nucleic acid-based alteration, DNA methylation, is associated with lung cancer. Accordingly, in one embodiment of the invention, the RNA isolated from mouth epithelial cells can be used for gene expression profiling. In another embodiment, the DNA isolated from mouth epithelial cells can be used for DNA methylation analysis.

One aspect of the invention provides biomarkers, also known as target genes, useful for the detection of lung cancer, or for assessing an individual's risk for developing lung cancer. The invention provides a method for detecting the expression of a target gene(s) of interest in a sample of buccal mucosa epithelial cells, comprising: isolating a nucleic acid sample from buccal mucosa epithelial cells, as described; contacting the isolated nucleic acid sample of step (a) with at least one nucleic acid probe which specifically hybridizes to the target gene(s) of interest; and detecting the presence of said target gene(s) of interest in the nucleic acid sample. In one embodiment, the target gene(s) of interest is attached to a solid phase prior to performing step (b). Preferably the nucleic acid is RNA or DNA.

The methods of the present invention can be used to identify target genes, or biomarkers, which are altered in the mouth epithelial cells of individuals having or at risk of developing a lung disorder.

Useful biomarkers include genes which are expressed at higher or lower levels in the mouth epithelial cells of individuals having or at risk of developing a lung disorder.

Specific examples of genes which are expressed in higher levels in the mouth epithelial cells of current smokers that they are expressed in people who have never smoked include ALDH3A1, CEACAM5, and NQO1, as illustrated in FIG. 4.

Other useful biomarkers are those which have different DNA patterns such as methylation patterns in the mouth epithelial cells of individuals having or at risk of developing a lung disorder. (Tsou et al., Oncogene 21:5450-5461 (2002); Fukami et al., Int. J. Cancer 107:53-59 (2003))

The present invention also provides the identification and characterization of “airway transcriptomes” or signature gene expression profiles of the airways and identification of changes in this transcriptome that are associated with epithelial exposure to pollutants, such as direct or indirect exposure to cigarette smoke, asbestos, and smog. A particularly preferred airway transcriptome is a mouth transcriptome, comprising genes whose expression differs significantly between the mouth epithelial cells of healthy smokers and healthy non-smokers. These airway transcriptome gene expression profiles provide information on lung tissue function upon cessation from smoking, predisposition to lung cancer in non-smokers and smokers, and predisposition to other lung diseases. The mouth transcriptome expression pattern can be obtained from a non-smoker, wherein deviations in the normal expression pattern are indicative of increased risk of lung diseases. The mouth transcriptome expression pattern can also be obtained from a non-smoking subject exposed to air pollutants, wherein deviation in the expression pattern associated with normal response to the air pollutants is indicative of increased risk of developing lung disease.

The present invention also provides a mouth transcriptome comprising a group consisting of genes encoding ABCC1; ABHD2; AF333388.1; AGTPBP1; AIP1; AKR1B10AKR1C1; AKR1C2; AL117536.1; AL353759; ALDH3A1; ANXA3; APLP2; ARHE; ARL1; ARPC3; ASM3A; B4GALT5; BECN1; C1orf8; C20orf111; C5orf6; C6orf80; CA12, CABYR; CANX; CAP1; CCNG2; CEACAM5; CEACAM6; CED-6; CHP; CHST4; CKB; CLDN10; CNK1; COPB2; COX5A; CPNE3; CRYM; CSTA; CTGF; CYP1B1; CYP2A6; CYP4F3; DEFB1; DIAPH2; DKFZP434J214; DKFZP564K0822; DKFZP566E144; DSCR5; DSG2; EPAS1; EPOR; FKBP1A; FLJ10134; FLJ13052; FLJ130521; FLJ20359; FMO2; FTH1; GALNT1; GALNT3; GALNT7; GCLC; GCLM; GGA1; GHITM; GMDS; GNE; GPX2; GRP58; GSN; GSTM3; GSTM5; GUK1; HIG1; HIST1H2BK; HN1; HPGD; HRIHFB2122; HSPA2; IDH1; IDS; IMPA2; ITM2A; JTB; KATNB1; KDELR3; KIAA0397; KIAA0905; KLF4; KRT14; KRT15; LAMP2; LOC51186; LOC57228; LOC92482; LOC92689; LYPLA1; MAFG; ME1; MGC4342; MGLL; MT1E; MT1F; MT1G; MT1H; MT1X; MT2A; NCOR2; NKX3-1; NQO1; NUDT4; ORL1; P4HB; PEX14; PGD; PRDX1; PRDX4; PSMB5; PSMD14; PTP4A1; PTS; RAB11A; RAB2; RAB7; RAP1GA1; RNP24; RPN2; S1A10; S100A14; S100P; SCP2; SDR1; SHARP1; SLC17A5; SLC35A3; SORD; SPINT2; SQSTM1; SRPUL; SSR4; TACSTD2; TALDO1; TARS; TCF7L1; TIAM1; TJP2; TLE1; TM4SF1; TM4SF13; TMP21; TNFSF13; TNS; TRA1; TRIM16; TXN; TXNDC5; TXNL; TXNRD1; UBE2J1; UFD1L; UGT1A10; YF13H12; and ZNF463. Table 1 below lists the GenBank ID and GenBank description corresponding to the HUGO identification symbol (ID) presented in this list of genes.

TABLE 1

| GENBANK_ID | HUGO_ID | GENBANK_DESCRIPTION |
|---|---|---|
| NM_017781.1 | FLJ20359 | hypothetical protein FLJ20359 |
| NM_018004.1 | FLJ10134 | hypothetical protein FLJ10134 |
| AF078844.1 | MT1F | metallothionein 1F (functional) |
| NM_005951.1 | MT1H | metallothionein 1H |
| BC005894.1 | FMO2 | flavin containing monooxygenase 2 |
| AF182275.1 | CYP2A6 | “cytochrome P450, family 2, subfamily A, |
| | | polypeptide 6” |
| BF246115 | MT1F | metallothionein 1F (functional) |
| NM_005952.1 | MT1X | metallothionein 1X |
| NM_005950.1 | MT1G | metallothionein 1G |
| NM_001823.1 | CKB | “creatine kinase, brain” |
| NM_000860.1 | HPGD | hydroxyprostaglandin dehydrogenase 15-(NAD) |
| AL021786 | ITM2A | integral membrane protein 2A |
| L29008.1 | SORD | sorbitol dehydrogenase |
| NM_002275.1 | KRT15 | keratin 15 |
| AF333388.1 | na | hypothetical gene supported by S68948 |
| U56725.1 | HSPA2 | heat shock 70 kDa protein 2 |
| M10943 | MT1F | metallothionein 1F (functional) |
| BF217861 | MT1E | metallothionein 1E (functional) |
| AF052094.1 | EPAS1 | endothelial PAS domain protein 1 |
| X97671 | EPOR | erythropoietin receptor |
| NM_002450.1 | MT1X | metallothionein 1X |
| AF114012.1 | TNFSF13 | “tumor necrosis factor (ligand) superfamily, |
| | | member 13” |
| NM_005953.1 | MT2A | metallothionein 2A |
| AL046979 | TNS | tensin |
| NM_000851.1 | GSTM5 | glutathione S-transferase M5 |
| AB017546 | PEX14 | peroxisomal biogenesis factor 14 |
| NM_006312.1 | NCOR2 | nuclear receptor co-repressor 2 |
| NM_006314.1 | CNK1 | connector enhancer of KSR-like |
| | | (Drosophila kinase suppressor of ras) |
| ABO14605.1 | AIP1 | atrophin-1 interacting protein 1 |
| NM_031283.1 | TCF7L1 | “transcription factor 7-like 1 |
| | | (T-cell specific, HMG-box)” |
| ABO07857 | KIAA0397 | KIAA0397 gene product |
| NM_001888.1 | CRYM | “crystallin, mu” |
| NM_005769.1 | CHST4 | carbohydrate (N-acetylglucosamine 6-O) |
| | | sulfotransferase 4 |
| BC0O6230.1 | MGLL | monoglyceride lipase |
| NM_018555.2 | ZNF463 | zinc finger protein 463 |
| NM_015001.1 | SHARP | SMART/HDAC1 associated repressor protein |
| NM_016605.1 | C5orf6 | chromosome 5 open reading frame 6 |
| AWO01443 | GGA1 | “golgi associated, gamma adaptin |
| | | ear containing, ARF binding protein 1” |
| AA046650 | HRIHFB2122 | Tara-like protein |
| Z97O56 | KDELR3 | KDEL (Lys-Asp-Glu-Leu) endoplasmic |
| | | reticulum protein retention receptor 3 |
| BC0O1049.1 | UFD1L | ubiquitin fusion degradation 1-like |
| NM_O15523.1 | DKFZP566E144 | small fragment nuclease |
| NM_O06694.1 | JTB | jumping translocation breakpoint |
| NM_O30796.1 | DKFZP564K0822 | Hypothetical protein DKFZp564K0822 |
| AF217514.1 | C20orf111 | chromosome 20 open reading frame 111 |
| AF027205.1 | SPINT2 | “serine protease inhibitor, Kunitz type, 2” |
| BC0O3379.1 | LOC57228 | Hypothetical protein from clone 643 |
| BC0O6249.1 | GUK1 | guanylate kinase 1 |
| NM_O04872.1 | C1orf8 | chromosome 1 open reading frame 8 |
| M94859.1 | CANX | Calnexin |
| NM_O00801.1 | FKBP1A | “FK506 binding protein 1A, 12 kDa” |
| AV7O6096 | LOC92482 | hypothetical protein LOC92482 |
| NM_O06367.2 | CAP1 | “CAP, adenylate cyclase-associated |
| | | protein 1 (yeast)” |
| AL556438 | TLE1 | “transducin regulation of transcription |
| | | DNA dependent |
| BC003560.1 | RPN2 | ribophorin II |
| NM_014297.1 | YF13H12 | protein expressed in thyroid |
| NM_003900.1 | SQSTM1 | sequestosome 1 |
| BC004146.1 | PSMB5 | “proteasome (prosome, macropain) |
| | | subunit, beta type, 5” |
| NM_004786.1 | TXNL | “thioredoxin-like, 32 kDa” |
| AI951720 | TLE1 | “transducin-like enhancer of split 1 |
| | | (E(sp1) homolog, Drosophila)” |
| NM_006280.1 | SSR4 | “signal sequence receptor, delta |
| | | (translocon-associated protein delta)” |
| NM_030810.1 | TXNDC5 | thioredoxin domain containing 5 |
| NM_004766.1 | COPB2 | “coatomer protein complex, |
| | | subunit beta 2 (beta prime)” |
| AF139131.1 | BECN1 | “beclin 1 (coiled-coil, myosin-like |
| | | BCL2 interacting protein)” |
| NM_006827.1 | TMP21 | transmembrane trafficking protein |
| NM_003299.1 | TRA1 | tumor rejection antigen (gp96) 1 |
| NM_020474.2 | GALNT1 | UDP-N-acetyl-alpha-D-galactosamine: |
| | | polypeptide N-acetylgalactosaminyltransferase |
| | | 1 (GalNAc-T1) |
| NM_005886.1 | KATNB1 | katanin p80 (WD repeat containing) |
| | | subunit B 1 |
| NM_024329.1 | MGC4342 | hypothetical protein MGC4342 |
| NM_004817.1 | TJP2 | tight junction protein 2 |
| | | (zona occludens 2) |
| AK000095.1 | CHP | calcium binding protein P22 |
| BC000758.1 | C6orf80 | chromosome 6 open reading frame 80 |
| AB035745.1 | DSCR5 | Down syndrome critical region gene 5 |
| NM_005805.1 | PSMD14 | “proteasome (prosome, macropain) |
| | | 26S subunit, non-ATPase, 14” |
| JO4152 | TACSTD2 | tumor-associated calcium |
| | | signal transducer 2 |
| NM_016021.1 | UBE2J1 | “ubiquitin-conjugating enzyme E2, |
| | | J1 (UBC6 homolog, yeast)” |
| BC004371.1 | APLP2 | amyloid beta (A4) precursor-like |
| | | protein 2 |
| NM_004255.1 | COX5A | cytochrome c oxidase subunit Va |
| AI215102 | RAB11A | “RAB11A, member RAS |
| | | oncogene family” |
| JO4183.1 | LAMP2 | lysosomal-associated |
| | | membrane protein 2 |
| NM_005896.1 | IDH1 | “isocitrate dehydrogenase 1 (NADP+), |
| | | soluble” |
| M97655.1 | PTS | 6-pyruvoyltetrahydropterin synthase |
| AK024976.1 | RNP24 | coated vesicle membrane protein |
| AF131820.1 | GHITM | growth hormone inducible |
| | | transmembrane protein |
| NM_000202.2 | IDS | iduronate 2-sulfatase |
| | | (Hunter syndrome) |
| NM_001177.2 | ARL1 | ADP-ribosylation factor-like 1 |
| AK000826.1 | RAB7 | “RAB7, member RAS |
| | | oncogene family” |
| NM_006406.1 | PRDX4 | peroxiredoxin 4 |
| D83485.1 | GRP58 | “glucose regulated protein, 58 kDa” |
| NM_014056.1 | HIG1 | likely ortholog of mouse hypoxia |
| | | induced gene 1 |
| NM_000177.1 | GSN | “gelsolin (amyloidosis, |
| | | Finnish type)” |
| BG054844 | ARHE | “ras homolog gene |
| | | family, member E” |
| BC001709.1 | FLJ13052 | NAD kinase |
| U90902.1 | TIAM1 | T-cell lymphoma invasion |
| | | and metastasis 1 |
| BC000893.1 | HIST1H2BK | “histone 1, H2bk” |
| AL353759 | — | “Homo sapiens histone 1, |
| | | H2ac, mRNA (cDNA clone |
| | | IMAGE: 6526471), partial cds” |
| NM_012434.1 | SLC17A5 | “solute carrier family 17 |
| | | (anion/sugar transporter), member 5” |
| AF004561.1 | ARPC3 | “actin related protein ⅔ |
| | | complex, subunit 3, 21 kDa” |
| NM_014933.1 | KIAA0905 | yeast Sec31p homolog |
| NM_003909.1 | CPNE3 | copine III |
| AW134535 | CCNG2 | cyclin G2 |
| BF031829 | DSG2 | desmoglein 2 |
| U48296.1 | PTP4A1 | “protein tyrosine phosphatase |
| | | type IVA, member 1” |
| NM_004776.1 | B4GALT5 | “UDP-Gal: betaGlcNAc beta |
| | | 1,4-galactosyltransferase, |
| | | polypeptide 5” |
| BC001709.1 | FLJ13052 | NAD kinase |
| NM_015239.1 | AGTPBP1 | ATP/GTP binding protein 1 |
| J02783.1 | P4HB | “procollagen-proline, |
| | | 2-oxoglutarate 4-dioxygenase |
| | | (proline 4-hydroxylase), beta polypeptide |
| | | (protein disulfide isomerase; |
| | | thyroid hormone binding protein p55)” |
| NM_020672.1 | S100A14 | S100 calcium binding protein A14 |
| AL527430 | GSTM3 | glutathione S-transferase M3 (brain) |
| NM_004753.1 | SDR1 | short-chain dehydrogenase/reductase 1 |
| NM_007011.1 | ABHD2 | abhydrolase domain containing 2 |
| AI539710 | ABCC1 | “ATP-binding cassette, |
| | | sub-family C (CFTR/MRP), member 1” |
| NM_002865.1 | RAB2 | “RAB2, member RAS oncogene family” |
| BG288007 | LYPLA1 | lysophospholipase I |
| NM_002032.1 | FTH1 | “ferritin, heavy polypeptide 1” |
| NM_002885.1 | RAP1GA1 | “RAP1, GTPase activating protein 1” |
| NM_006729.1 | DIAPH2 | diaphanous homolog 2 (Drosophila) |
| AF200715.1 | CED-6 | PTB domain adaptor protein CED-6 |
| BC005911.1 | SCP2 | sterol carrier protein 2 |
| BF063271 | GALNT3 | UDP-N-acetyl-alpha-D-galactosamine: |
| | | polypeptide N-acetylgalactosaminyltransferase |
| | | 3 (GalNAc-T3) |
| NM_014399.1 | TM4SF13 | transmembrane 4 superfamily member 13 |
| NM_005476.2 | GNE | UDP-N-acetylglucosamine-2- |
| | | epimerase/N-acetylmannosamine kinase |
| NM_019094.1 | NUDT4 | nudix (nucleoside diphosphate |
| | | linked moiety X)-type motif 4 |
| AI762113 | GMDS | “GDP-mannose 4,6-dehydratase” |
| NM_014214.1 | IMPA2 | inositol(myo)-1(or 4)-monophosphatase 2 |
| AV728268 | SORL1 | “sortilin-related receptor, L(DLR class) |
| | | A repeats-containing” |
| NM_003191.1 | TARS | threonyl-tRNA synthetase |
| NM_016303.1 | Xq22.2 | |
| NM_012243.1 | SLC35A3 | “solute carrier family 35 |
| | | (UDP-N-acetylglucosamine |
| | | (UDP-GlcNAc) transporter), member A3” |
| AA873600 | ASM3A | acid sphingomyelinase-like phosphodiesterase |
| W87466 | Loc92689 | Hypothetical protein bc001096 |
| NM_016315.1 | CED-6 | PTB domain adaptor protein CED-6 |
| AF247704.1 | NKX3-1 | “NK3 transcription factor related, locus 1 |
| | | (Drosophila)” |
| NM_001072.1 | UGT1A10 | “UDP glycosyltransferase 1 family, |
| | | polypeptide A10” |
| NM_002359.1 | MAFG | v-maf musculoaponeurotic |
| | | fibrosarcoma oncogene homolog G (avian) |
| NM_005980.1 | S100P | S100 calcium binding protein P |
| NM_000896.1 | CYP4F3 | “cytochrome P450, family 4, |
| | | subfamily F, polypeptide 3” |
| L19184.1 | PRDX1 | peroxiredoxin 1 |
| NM_002966.1 | S100A10 | “S100 calcium binding protein A10 |
| | | (annexin II ligand, calpactin I, light |
| | | polypeptide (p11))” |
| NM_021027.1 | UGT1A10 | “UDP glycosyltransferase 1 family, |
| | | polypeptide A10” |
| NM_017423.1 | GALNT7 | UDP-N-acetyl-alpha-D-galactosamine: |
| | | polypeptide N-acetylgalactosaminyltransferase |
| | | 7 (GalNAc-T7) |
| BF676980 | GCLC | “glutamate-cysteine ligase, |
| | | catalytic subunit” |
| NM_001500.1 | GMDS | “GDP-mannose 4,6-dehydratase” |
| NM_016185.1 | HN1 | hematological and neurological |
| | | expressed 1 |
| AA083483 | FTH1 | “ferritin, heavy polypeptide 1” |
| AL117536.1 | na | hypothetical gene supported by |
| | | AK057191; AL117536 |
| M92934.1 | CTGF | connective tissue growth factor |
| M63310.1 | ANXA3 | annexin A3 |
| NM_000463.1 | UGT1A10 | “UDP glycosyltransferase |
| | | 1 family, polypeptide A10” |
| NM_001218.2 | CA12 | carbonic anhydrase XII |
| NM_012189.1 | CABYR | calcium-binding tyrosine-(Y)- |
| | | phosphorylation regulated (fibrousheathin 2) |
| BC005008.1 | CEACAM6 | carcinoembryonic antigen-related |
| | | cell adhesion molecule 6 |
| | | (non-specific cross reacting antigen) |
| NM_003330.1 | TXNRD1 | thioredoxin reductase 1 |
| NM_002631.1 | PGD | phosphogluconate dehydrogenase |
| NM_002061.1 | GCLM | “glutamate-cysteine ligase, modifier subunit” |
| NM_006755.1 | TALDO1 | transaldolase 1 |
| M18728.1 | CEACAM6 | carcinoembryonic antigen-related |
| | | cell adhesion molecule 6 |
| | | (non-specific cross reacting antigen) |
| NM_005213.1 | CSTA | cystatin A (stefin A) |
| U73945.1 | DEFB1 | “defensin, beta 1” |
| AF313911.1 | TXN | Thioredoxin |
| BF514079 | KLF4 | Kruppel-like factor 4 (gut) |
| NM_006470.1 | TRIM16 | tripartite motif-containing 16 |
| NM_014467.1 | SRPUL | sushi-repeat protein |
| AL049699 | ME1 | “malic enzyme 1, NADP(+)-dependent, |
| | | cytosolic” |
| NM_002395.2 | ME1 | “malic enzyme 1, NADP(+)-dependent, |
| | | cytosolic” |
| BC002690.1 | KRT14 | “keratin 14 (epidermolysis bullosa simplex, |
| | | Dowling-Meara, Koebner)” |
| AI346835 | TM4SF1 | transmembrane 4 superfamily member 1 |
| NM_001353.2 | AKR1C1 | “aldo-keto reductase family 1, member C1 |
| | | (dihydrodiol dehydrogenase 1; 20-alpha |
| | | (3-alpha)-hydroxysteroid dehydrogenase)” |
| BC000906.1 | NQO1 | “NAD(P)H dehydrogenase, quinone 1” |
| NM_006984.1 | CLDN10 | claudin 10 |
| S68290.1 | AKR1C1 | “aldo-keto reductase family 1, |
| | | member C1 (dihydrodiol |
| | | dehydrogenase 1; 20-alpha |
| | | (3-alpha)-hydroxysteroid dehydrogenase)” |
| M33376.1 | AKR1C2 | “aldo-keto reductase family 1, |
| | | member C2 (dihydrodiol dehydrogenase 2; |
| | | bile acid binding protein; 3-alpha |
| | | hydroxysteroid dehydrogenase, type III)” |
| NM_002083.1 | GPX2 | glutathione peroxidase 2 (gastrointestinal) |
| NM_000903.1 | NQO1 | “NAD(P)H dehydrogenase, quinone 1” |
| NM_000691.1 | ALDH3A1 | “aldehyde dehydrogenase 3 family, |
| | | memberA1” |
| NM_004363.1 | CEACAM5 | carcinoembryonic antigen-related |
| | | cell adhesion molecule 5 |
| NM_000104.2 | CYP1B1 | “cytochrome P450, family 1, |
| | | subfamily B, polypeptide 1” |
| NM_020299.1 | AKR1B10 | “aldo-keto reductase family 1, |
| | | member B10 (aldose reductase)” |

In one preferred embodiment, the invention provides a mouth transcriptome comprising a group consisting of genes encoding: AGTPBP1; AKR1C1; AKR1C2; ALDH3A1; ANXA3; CA12; CEACAM6; CLDN10; CYP1B1; DPYSL3; FLJ13052; FTH1; GALNT3; GALNT7; GCLC; GCLM; GMDS; GPX2; HN1; HSPA2; MAFG; ME1; MGLL; MMP10; MT1F; MT1G; MT1X; NQO1; NUDT4; PGD; PRDX1; PRDX4; RAB11A; S10A10; SDR1; SRPUL; TALDO1; TARS; TCF-3; TRA1; TRIM16; and TXN. Table 2 below lists the GenBank ID and GenBank description corresponding to the HUGO identification symbol (ID) presented in this list of genes.

TABLE 2

| AFFX | GENBANK | HUGO | GO | GENBANK |
|---|---|---|---|---|
| ID | ID | ID | ID | DESCRIPTION |
| 205680_at | NM_002425 | MMP10 | 30574 | matrix metalloproteinase 10 |
| | | | | (stromelysin 2) |
| 210524_x_at | NM_007372 | MT1F | 5737 | RNA helicase-related protein |
| 208581_x_at | NM_005952 | MT1X | 9634 | metallothionein 1X |
| 211538_s_at | NM_021979 | HSPA2 | 7286 | heat shock 70 kD protein 2 |
| 204745_x_at | NM_005950 | MT1G | 46872 | metallothionein 1G |
| 217165_x_at | M10943 | MT1F | 5737 | |
| 221016_s_at | NM_031283 | TCF-3 | 6355 | HMG-box transcription |
| | | | | factor TCF-3 |
| 211026_s_at | NM_007283 | MGLL | 6954 | monoglyceride lipase |
| 200599_s_at | NM_003299 | TRA1 | 5524 | tumor rejection antigen |
| | | | | (gp96) 1 |
| 200863_s_at | NM_004663 | RAB11A | 6886 | RAB11A, member |
| | | | | RAS oncogene family |
| 201923_at | NM_006406 | PRDX4 | 7252 | peroxiredoxin 4 |
| 208918_s_at | NM_023018 | FLJ13052 | | NAD kinase |
| 208919_s_at | NM_023018 | FLJ13052 | | NAD kinase |
| 202481_at | NM_004753 | SDR1 | 8152 | short-chain |
| | | | | dehydrogenase/reductase 1 |
| 204500_s_at | NM_015239 | AGTPBP1 | | ATP/GTP binding |
| | | | | protein 1 |
| 206302_s_at | NM_019094 | NUDT4 | 9187 | nudix (nucleoside |
| | | | | diphosphate |
| | | | | linked moiety X)-type |
| | | | | motif 4 |
| 200748_s_at | NM_002032 | FTH1 | 6826 | ferritin, heavy |
| | | | | polypeptide 1 |
| 203397_s_at | NM_004482 | GALNT3 | 5975 | UDP-N-acetyl- |
| | | | | alpha-D-galactosamine: |
| | | | | polypeptide |
| | | | | N-acetylgalactosaminyl |
| | | | | transferase 3 |
| | | | | (GalNAc-T3) |
| 214106_s_at | NM_001500 | GMDS | 5975 | GDP-mannose |
| | | | | 4,6-dehydratase |
| 201263_at | NM_003191 | TARS | 6435 | threonyl-tRNA |
| | | | | synthetase |
| 204970_s_at | NM_002359 | MAFG | 6355 | v-maf musculoaponeurotic |
| | | | | fibrosarcoma oncogene |
| | | | | homolog G (avian) |
| 200872_at | NM_002966 | S100A10 | 7165 | S100 calcium |
| | | | | binding protein A10 |
| | | | | (annexin II ligand, |
| | | | | calpactin I, light |
| | | | | polypeptide (p11)) |
| 208680_at | NM_002574 | PRDX1 | 8283 | peroxiredoxin 1 |
| 218313_s_at | NM_017423 | GALNT7 | 5975 | UDP-N-acetyl-alpha- |
| | | | | D-galactosamine: |
| | | | | polypeptide |
| | | | | N-acetylgalactosaminyl |
| | | | | transferase 7 (GalNAc-T7) |
| 201431_s_at | NM_001387 | DPYSL3 | 7165 | dihydropyrimidinase-like 3 |
| 217755_at | NM_016185 | HN1 | | hematological and |
| | | | | neurological expressed 1 |
| 203963_at | NM_001218 | CA12 | 6730 | carbonic anhydrase XII |
| 202923_s_at | NM_001498 | GCLC | 6534 | glutamate-cysteine |
| | | | | ligase, catalytic subunit |
| 204875_s_at | NM_001500 | GMDS | 5975 | GDP-mannose |
| | | | | 4,6-dehydratase |
| 201266_at | NM_003330 | TXNRD1 | 6118 | thioredoxin reductase 1 |
| 201118_at | NM_002631 | PGD | 9051 | phosphogluconate |
| | | | | dehydrogenase |
| 209369_at | NM_005139 | ANXA3 | 5737 | annexin A3 |
| 203925_at | NM_002061 | GCLM | 6534 | glutamate-cysteine |
| | | | | ligase, modifier subunit |
| 211657_at | M18728.1 | CEACAM6 | 7165 | |
| 208864_s_at | NM_003329 | TXN | 7165 | thioredoxin |
| 201463_s_at | NM_006755 | TALDO1 | 5975 | transaldolase 1 |
| 203757_s_at | NM_002483 | CEACAM6 | 7165 | carcinoembryonic antigen- |
| | | | | related cell adhesion |
| | | | | molecule 6 (non-specific |
| | | | | cross reacting antigen) |
| 205499_at | NM_014467 | SRPUL | 6118 | sushi-repeat protein |
| 204341_at | NM_006470 | TRIM16 | 5737 | tripartite motif-containing 16 |
| 204058_at | AL049699 | ME1 | 6099 | |
| 221841_s_at | NM_004235 | — | | Kruppel-like |
| | | | | factor 4 (gut) |
| 204059_s_at | NM_002395 | ME1 | 6099 | malic enzyme 1, |
| | | | | NADP(+)-dependent, |
| | | | | cytosolic |
| 204151_x_at | NM_001353 | AKR1C1 | 6805 | aldo-keto reductase |
| | | | | family 1, member C1 |
| | | | | (dihydrodiol dehydrogenase |
| | | | | 1; 20-alpha (3-alpha)- |
| | | | | hydroxysteroid dehydrogenase) |
| 210519_s_at | BC000906.1 | NQO1 | 6118 | |
| 216594_x_at | S68290.1 | AKR1C1 | 6805 | |
| 202831_at | NM_002083 | GPX2 | 6979 | glutathione peroxidase 2 |
| | | | | (gastrointestinal) |
| 205328_at | NM_006984 | CLDN10 | 7155 | claudin 10 |
| 201468_s_at | NM_000903 | NQO1 | 6118 | NAD(P)H |
| | | | | dehydrogenase, quinone 1 |
| 201467_s_at | NM_000903 | NQO1 | 6118 | NAD(P)H dehydrogenase, |
| | | | | quinone 1 |
| 209699_x_at | NM_001354 | AKR1C2 | 15722 | aldo-keto reductase |
| | | | | family 1, member C2 |
| | | | | (dihydrodiol dehydrogenase 2; |
| | | | | bile acid binding protein; |
| | | | | 3-alpha hydroxysteroid |
| | | | | dehydrogenase, type III) |
| 217626_at | BF508244 | AKR1C1 | 6805 | ESTs, Highly similar to |
| | | | | DBDD_HUMAN |
| | | | | TRANS-1,2- |
| | | | | DIHYDROBENZENE-1,2- |
| | | | | DIOL DEHYDROGENASE |
| | | | | [H. sapiens] |
| 205623_at | NM_000691 | ALDH3A1 | 6081 | aldehyde dehydrogenase |
| | | | | 3 family, memberA1 |
| 202435_s_at | NM_000104 | CYP1B1 | 6118 | cytochrome P450, |
| | | | | subfamily I (dioxin-inducible), |
| | | | | polypeptide 1 (glaucoma 3, |
| | | | | primary infantile) |
| 202436_s_at | NM_000104 | CYP1B1 | 6118 | cytochrome P450, |
| | | | | subfamily I (dioxin-inducible), |
| | | | | polypeptide 1 (glaucoma 3, |
| | | | | primary infantile) |
| 202437_s_at | NM_000104 | CYP1B1 | 6118 | cytochrome P450, |
| | | | | subfamily I (dioxin-inducible), |
| | | | | polypeptide 1 (glaucoma 3, |
| | | | | primary infantile) |

The present invention contemplates use of its methods to identify mouth transcriptomes, unique sets of expressed genes, or gene expression patterns in mouth epithelial cells associated with pre-malignancy in the lung and lung cancer in smokers and non-smokers. All of these expression patterns constitute expression signatures that indicate operability and pathways of cellular function that can be used to guide decisions regarding prognosis, diagnosis and possible therapy. Epithelial cell gene expression profiles obtained from relatively accessible sites such as the mouth can thus provide important prognostic, diagnostic, and therapeutic information which can be applied to diagnose and treat lung disorders.

Accordingly, in one embodiment, the invention provides a “mouth transcriptome” the expression pattern of which is useful in screening, prognostic, diagnostic and therapeutic applications as described herein.

Techniques of the present invention include detection with nucleotide probes. Preferably, the nucleotide probes may be any that will selectively hybridize to a target gene of interest. For example, it will hybridize to the target gene transcript more strongly than to other naturally occurring transcription factor sequences. Types of probes include cDNA, riboprobes, synthetic oligonucleotides and genomic probe. The type of probe used will generally be dictated by the particular situation, such as riboprobes for in situ hybridization, and cDNA for Northern blotting, for example. Detection of the target encoding gene, per se, will be useful in screening for conditions associated with enhanced expression. Other forms of assays to detect targets more readily associated with levels of expression-transcripts and other expression products will generally be useful as well. The probes may be as short as is required to differentially recognize mRNA transcripts of interest, and may be as short as, for example, 15 bases, more preferably it is at least 17 bases. Still more preferably the probe is at least 20 bases.

A probe may also be reverse-engineered by one skilled in the art from the amino acid sequence of the target gene. However use of such probes may be limited, as it will be appreciated that any one given reverse-engineered sequence will not necessarily hybridize well, or at all with any given complementary sequence reverse-engineered from the same peptide, owing to the degeneracy of the genetic code. This is a factor common in the calculations of those skilled in the art, and the degeneracy of any given sequence is frequently so broad as to yield a large number of probes for any one sequence.

The form of labeling of the probes may be any that is appropriate, such as the use of radioisotopes, for example, ³²P and ³⁵S. Labeling with radioisotopes may be achieved, whether the probe is synthesized chemically or biologically, by the use of suitably labeled bases. Other forms of labeling may include enzyme or antibody labeling such as is characteristic of ELISA, or any reporter molecule. A “reporter molecule”, as used herein, is a molecule which provides an analytically identifiable signal allowing detection of a hybridized probe. Detection may be either qualitative or quantitative. Commonly used reporter molecules include fluorophores, enzymes, biotin, chemiluminescent molecules, bioluminescent molecules, digoxigenin, avidin, streptavidin, or radioisotopes. Commonly used enzymes include horseradish peroxidase, alkaline phosphatase, glucose oxidase and beta-galactosidase, among others. Enzymes can be conjugated to avidin or streptavidin for use with a biotinylated probe. Similarly, probes can be conjugated to avidin or streptavidin for use with a biotinylated enzyme. The substrates to be used with these enzymes are generally chosen for the production, upon hydrolysis by the corresponding enzyme, of a detectable color change. For example, p-nitrophenyl phosphate is suitable for use with alkaline phosphatase reporter molecules; for horseradish peroxidase, 1,2-phenylenediamine, 5-aminosalicylic acid or tolidine are commonly used. Incorporation of a reporter molecule into a DNA probe can be by any method known to the skilled artisan, for example by nick translation, primer extension, random oligo priming, by 3′ or 5′ end labeling or by other means (see, for example, Sambrook et al. Molecular Biology: A laboratory Approach, Cold Spring Harbor, N.Y. 1989).

### Detection of Gene Expression

In one embodiment of the present invention, the isolated epithelial nucleic acid can be used to evaluate expression of a gene or multiple genes using any method known in the art for measuring gene expression, including analysis of mRNA transcripts as well as analysis of DNA methylation.

Methods for assessing mRNA levels are well known to those skilled in the art. In one preferred embodiment, gene expression can be determined by detection of RNA transcripts, for example by Northern blotting, for example, wherein a preparation of RNA is run on a denaturing agarose gel, and transferred to a suitable support, such as activated cellulose, nitrocellulose or glass or nylon membranes. Labeled (e.g. radiolabeled) cDNA or RNA is then hybridized to the preparation, washed and analyzed using methods well known in the art, such as autoradiography.

Detection of RNA transcripts can further be accomplished using known amplification methods. For example, it is within the scope of the present invention to reverse transcribe mRNA into cDNA followed by polymerase chain reaction (RT-PCR); or, to use a single enzyme for both steps as described in U.S. Pat. No. 5,322,770, or reverse transcribe mRNA into cDNA followed by symmetric gap ligase chain reaction (RT-AGLCR) as described by R. L. Marshall, et al., PCR Methods and Applications 4: 80-84 (1994).

Other known amplification methods which can be utilized herein include but are not limited to the so-called “NASBA” or “3SR” technique described in PNAS USA 87: 1874-1878 (1990) and also described in Nature 350 (No. 6313): 91-92 (1991); Q-beta amplification as described in published European Patent Application (EPA) No. 4544610; strand displacement amplification (as described in G. T. Walker et al., Clin. Chem. 42: 9-13 (1996) and European Patent Application No. 684315; and target mediated amplification, as described by PCT Publication WO 9322461.

In situ hybridization visualization may also be employed, wherein a radioactively labeled antisense RNA probe is hybridized with a thin section of a biopsy sample, washed, cleaved with RNase and exposed to a sensitive emulsion for autoradiography. The samples may be stained with haematoxylin to demonstrate the histological composition of the sample, and dark field imaging with a suitable light filter shows the developed emulsion. Non-radioactive labels such as digoxigenin may also be used.

Alternatively, RNA expression, including mRNA expression, can be detected on a DNA array, chip or a microarray. Oligonucleotides corresponding to a gene(s) of interest are immobilized on a chip which is then hybridized with labeled nucleic acids of a test sample obtained from a patient. Positive hybridization signal is obtained with the sample containing transcripts of the gene of interest. Methods of preparing DNA arrays and their use are well known in the art. (See, for example U.S. Pat. Nos. 6,618,6796; 6,379,897; 6,664,377; 6,451,536; 548,257; U.S. 2003 0157485 and Schena et al. 1995 Science 20:467-470; Gerhold et al. 1999 Trends in Biochem. Sci. 24, 168-173; and Lennon et al. 2000 Drug discovery Today 5: 59-65, which are herein incorporated by reference in their entirety). Serial Analysis of Gene Expression (SAGE) can also be performed (See for example U.S. Patent Application 20030215858).

The methods of the present invention can employ solid substrates, including arrays in some preferred embodiments. Methods and techniques applicable to polymer array synthesis have been described in U.S. Ser. No. 09/536,841, WO 00/58516, U.S. Pat. Nos. 5,143,854, 5,242,974, 5,252,743, 5,324,633, 5,384,261, 5,405,783, 5,424,186, 5,451,683, 5,482,867, 5,491,074, 5,527,681, 5,550,215, 5,571,639, 5,578,832, 5,593,839, 5,599,695, 5,624,711, 5,631,734, 5,795,716, 5,831,070, 5,837,832, 5,856,101, 5,858,659, 5,936,324, 5,968,740, 5,974,164, 5,981,185, 5,981,956, 6,025,601, 6,033,860, 6,040,193, 6,090,555, 6,136,269, 6,269,846 and 6,428,752, in PCT Applications Nos. PCT/US99/00730 (International Publication Number WO 99/36760) and PCT/US01/04285, which are all incorporated herein by reference in their entirety for all purposes.

Patents that describe synthesis techniques in specific embodiments include U.S. Pat. Nos. 5,412,087, 6,147,205, 6,262,216, 6,310,189, 5,889,165, and 5,959,098.

Nucleic acid arrays that are useful in the present invention include, but are not limited to those that are commercially available from Affymetrix (Santa Clara, Calif.) under the brand name GeneChip7. Example arrays are shown on the website at affymetrix.com.

The present invention also contemplates many uses for polymers attached to solid substrates. These uses include gene expression monitoring, profiling, library screening, genotyping and diagnostics. Examples of gene expression monitoring, and profiling methods are shown in U.S. Pat. Nos. 5,800,992, 6,013,449, 6,020,135, 6,033,860, 6,040,138, 6,177,248 and 6,309,822. Examples of genotyping and uses therefore are shown in U.S. Ser. No. 60/319,253, 10/013,598, and U.S. Pat. Nos. 5,856,092, 6,300,063, 5,858,659, 6,284,460, 6,361,947, 6,368,799 and 6,333,179. Other examples of uses are embodied in U.S. Pat. Nos. 5,871,928, 5,902,723, 6,045,996, 5,541,061, and 6,197,506.

To monitor mRNA levels, for example, mRNA is extracted from the biological sample to be tested, reverse transcribed, and fluorescent-labeled cDNA probes are generated. The microarrays capable of hybridizing to the gene of interest are then probed with the labeled cDNA probes, the slides scanned and fluorescence intensity measured. This intensity correlates with the hybridization intensity and expression levels.

In one preferred embodiment, gene expression is measured using quantitative real time PCR. Quantitative real-time PCR refers to a polymerase chain reaction which is monitored, usually by fluorescence, over time during the amplification process, to measure a parameter related to the extent of amplification of a particular sequence. The amount of fluorescence released during the amplification cycle is proportional to the amount of product amplified in each PCR cycle.

The present invention also contemplates many uses for polymers attached to solid substrates. These uses include gene expression monitoring, profiling, library screening, genotyping and diagnostics. Examples of gene expression monitoring, and profiling methods are shown in U.S. Pat. Nos. 5,800,992, 6,013,449, 6,020,135, 6,033,860, 6,040,138, 6,177,248 and 6,309,822. Examples of genotyping and uses therefore are shown in U.S. Ser. No. 60/319,253, 10/013,598, and U.S. Pat. Nos. 5,856,092, 6,300,063, 5,858,659, 6,284,460, 6,361,947, 6,368,799 and 6,333,179. Other examples of uses are embodied in U.S. Pat. Nos. 5,871,928, 5,902,723, 6,045,996, 5,541,061, and 6,197,506.

The present invention also contemplates sample preparation methods in certain preferred embodiments. Prior to or concurrent with expression analysis, the nucleic acid sample may be amplified by a variety of mechanisms, some of which may employ PCR. See, e.g., PCR Technology: Principles and Applications for DNA Amplification (Ed. H. A. Erlich, Freeman Press, NY, N.Y., 1992); PCR Protocols: A Guide to Methods and Applications (Eds. Innis, et al., Academic Press, San Diego, Calif., 1990); Mattila et al., Nucleic Acids. Res. 19, 4967 (1991); Eckert et al., PCR Methods and Applications 1, 17 (1991); PCR (Eds. McPherson et al., IRL Press, Oxford); and U.S. Pat. Nos. 4,683,202, 4,683,195, 4,800,159 4,965,188, and 5,333,675, and each of which is incorporated herein by reference in their entireties for all purposes. The sample may be amplified on the array. See, for example, U.S. Pat. No. 6,300,070 and U.S. patent application Ser. No. 09/513,300, which are incorporated herein by reference.

Other suitable amplification methods include the ligase chain reaction (LCR) (e.g., Wu and Wallace, Genomics 4, 560 (1989), Landegren et al., Science 241, 1077 (1988) and Barringer et al. Gene 89:117 (1990)), transcription amplification (Kwoh et al., Proc. Natl. Acad. Sci. USA 86, 1173 (1989) and WO88/10315), self-sustained sequence replication (Guatelli et al., Proc. Nat. Acad. Sci. USA, 87, 1874 (1990) and WO90/06995), selective amplification of target polynucleotide sequences (U.S. Pat. No. 6,410,276), consensus sequence primed polymerase chain reaction (CP-PCR) (U.S. Pat. No. 4,437,975), arbitrarily primed polymerase chain reaction (AP-PCR) (U.S. Pat. Nos. 5,413,909, 5,861,245) and nucleic acid based sequence amplification (NABSA). (See, U.S. Pat. Nos. 5,409,818, 5,554,517, and 6,063,603, each of which is incorporated herein by reference). Other amplification methods that may be used are described in, U.S. Pat. Nos. 5,242,794, 5,494,810, 4,988,617 and in U.S. Ser. No. 09/854,317, each of which is incorporated herein by reference.

Additional methods of sample preparation and techniques for reducing the complexity of a nucleic sample are described, for example, in Dong et al., Genome Research 11, 1418 (2001), in U.S. Pat. Nos. 6,361,947, 6,391,592 and U.S. patent application Ser. Nos. 09/916,135, 09/920,491, 09/910,292, and 10/013,598.

Methods for conducting polynucleotide hybridization assays have been well developed in the art. Hybridization assay procedures and conditions will vary depending on the application and are selected in accordance with the general binding methods known including those referred to in: Maniatis et al. Molecular Cloning: A Laboratory Manual (2^{nd} Ed. Cold Spring Harbor, N.Y., 1989); Berger and Kimmel Methods in Enzymology, Vol. 152, Guide to Molecular Cloning Techniques(Academic Press, Inc., San Diego, Calif., 1987); Young and Davism, P.N.A.S, 80; 1194 (1983). Methods and apparatus for carrying out repeated and controlled hybridization reactions have been described, for example, in U.S. Pat. Nos. 5,871,928, 5,874,219, 6,045,996 and 6,386,749, 6,391,623 each of which are incorporated herein by reference.

The present invention also contemplates signal detection of hybridization between ligands in certain preferred embodiments. See, for example, U.S. Pat. Nos. 5,143,854, 5,578,832; 5,631,734; 5,834,758; 5,936,324; 5,981,956; 6,025,601; 6,141,096; 6,185,030; 6,201,639; 6,218,803; and 6,225,625, in provisional U.S. Patent application 60/364,731 and in PCT Application PCT/US99/06097 (published as WO99/47964), each of which also is hereby incorporated by reference in its entirety for all purposes.

Examples of methods and apparatus for signal detection and processing of intensity data are disclosed in, for example, U.S. Pat. Nos. 5,143,854, 5,547,839, 5,578,832, 5,631,734, 5,800,992, 5,834,758; 5,856,092, 5,902,723, 5,936,324, 5,981,956, 6,025,601, 6,090,555, 6,141,096, 6,185,030, 6,201,639; 6,218,803; and 6,225,625, in U.S. Patent application 60/364,731 and in PCT Application PCT/US99/06097 (published as WO99/47964), each of which also is hereby incorporated by reference in its entirety for all purposes.

The practice of the present invention may also employ conventional biology methods, software and systems. Computer software products of the invention typically include computer readable medium having computer-executable instructions for performing the logic steps of the method of the invention. Suitable computer readable medium include floppy disk, CD-ROM/DVD/DVD-ROM, hard-disk drive, flash memory, ROM/RAM, magnetic tapes and etc. The computer executable instructions may be written in a suitable computer language or combination of several languages. Basic computational biology methods are described in, e.g. Setubal and Meidanis et al., Introduction to Computational Biology Methods (PWS Publishing Company, Boston, 1997); Salzberg, Searles, Kasif, (Ed.), Computational Methods in Molecular Biology, (Elsevier, Amsterdam, 1998); Rashidi and Buehler, Bioinformatics Basics: Application in Biological Science and Medicine(CRC Press, London, 2000) and Ouelette and Bzevanis Bioinformatics A Practical Guide for Analysis of Gene and Proteins (Wiley & Sons, Inc., 2nd ed., 2001).

The present invention also makes use of various computer program products and software for a variety of purposes, such as probe design, management of data, analysis, and instrument operation. See, for example, U.S. Pat. Nos. 5,593,839, 5,795,716, 5,733,729, 5,974,164, 6,066,454, 6,090,555, 6,185,561, 6,188,783, 6,223,127, 6,229,911 and 6,308,170.

Additionally, the present invention may have preferred embodiments that include methods for providing genetic information over networks such as the Internet as shown in, for example, U.S. patent application Ser. Nos. 10/063,559, 60/349,546, 60/376,003, 60/394,574, 60/403,381.

Throughout this specification, various aspects of this invention are presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range. In addition, the fractional ranges are also included in the exemplified amounts that are described. Therefore, for example, a range between 1-3 includes fractions such as 1.1, 1.2, 1.3, 1.4, 1.5, 1.6, etc.

### Differential DNA Methylation

The present invention provides methods to analyze DNA methylation patterns which are specifically associated with a gene in the mouth epithelial cells of a healthy individual, as compared to an individual having or at risk of developing lung disorders. Such differential methylation can be detected an enzyme that selectively cleaves only a differential DNA recognition site. For example, digesting DNA with an enzyme that cleaves only at a DNA recognition site that is methylated or by digesting with an enzyme that cleaves only at a DNA recognition site that is unmethylated. Any enzyme that is capable of selectively cleaving DNA regions from a healthy individual and not the corresponding DNA regions of an individual having or at risk of developing a lung disorder is useful in the present invention.

As used herein, “methyl-sensitive” enzymes are DNA restriction endonucleases that are dependent on the methylation state of their DNA recognition site for activity. For example, there are methyl-sensitive enzymes that cleave at their DNA recognition sequence only if it is not methylated. Thus, an unmethylated DNA sample will be cut into smaller sizes than a methylated DNA sample. Similarly, a hypermethylated DNA sample will not be cleaved and will give rise to larger fragments than a normally non-methylated DNA sample. In contrast, there are methyl-sensitive enzymes that cleave at their DNA recognition sequence only if it is methylated. As used herein, the terms “cleave”, “cut” and “digest” are used interchangeably.

Methyl-sensitive enzymes that digest unmethylated DNA suitable for use in methods of the invention include, but are not limited to, HpaII, HhaI, MaeII, BstUI and AciI. A preferred enzyme of use is HpaII that cuts only the unmethylated sequence CCGG. Combinations of methyl-sensitive enzymes that digest only unmethylated DNA can also be used. Suitable enzymes that digest only methylated DNA include, but are not limited to, DpnI and McrBC (New England BioLabs).

DNA that is obtained from a buccal epithelial cell sample can be isolated by any standard means known to a skilled artisan. Standard methods of DNA isolation are described in Sambrook et al., Molecular Biology: A laboratory Approach, Cold Spring Harbor, N.Y. 1989; Ausubel, et al., Current protocols in Molecular Biology, Greene Publishing, Y, 1995.

Cleavage methods and procedures for selected restriction enzymes for cutting DNA at specific sites are known to the skilled artisan. For example, many suppliers of restriction enzymes provide information on conditions and types of DNA sequences cut by specific restriction enzymes, including New England BioLabs, Pro-Mega Biochems, Boehringer-Mannheim and the like. Sambrook et al. (See Sambrook et al., Molecular Biology: A laboratory Approach, Cold Spring Harbor, N.Y. 1989) provide a general description of methods for using restriction enzymes and other enzymes. In the methods of the present invention it is preferred that the enzymes are used under conditions that will enable cleavage of DNA with 95%-100% efficiency.

Identification of Methyl-Polymorphic Probes that Detect Differentially Methylated DNA

The present invention exploits differences in healthy and non-healthy DNA as a means to identify methyl-polymorphic probes. In one embodiment, the invention exploits differential methylation. In mammalian cells, methylation plays an important role in gene expression. For example, genes (promoter and first exon region) are frequently not methylated in cells where they are expressed and are methylated in cell types where they are not expressed. It is known that methylation alterations are common occurrences in lung cancer. (Tsou et al., 2002). DNA fragments which represent regions of differential methylation can be sequenced and screened for the presence of polymorphic markers which can be used as biomarkers for the present invention. Polymorphic markers can be found in public databases, such as NCBI, or discovered by sequencing. The identified methyl-polymorphic markers can then used as a diagnostic of chromosomal abnormalities by assessing their correlation in healthy individuals as compared to individuals having or at risk of developing lung disorders, such as lung cancer.

Regions of differential methylation can be identified by any means known in the art and probes and/or primers corresponding to those regions accordingly prepared. Various methods for identifying regions of differential methylation are described in U.S. Pat. Nos. 5,871,917, 5,436,142 and U.S. Application No.'s 20020155451A1 and US20030022215A1, US20030099997, the contents of which are herein incorporated by reference.

Examples of how to identify regions of that are differentially methylated in healthy individuals as compared to individuals having or at risk of developing lung disorders, such as lung cancer DNA follow.

One method is described in U.S. Pat. No. 5,871,917. The method detects differential methylation at CpNpG sequences by cutting test DNA control DNA with a CNG specific restriction enzyme that does not cut methylated DNA. The method uses one or more rounds of DNA amplification coupled with subtractive hybidization to identify differentially methylated or mutated segments of DNA. Thus, the method can selectively identify regions of the genome that are hypo- or hypermethylated.

A Southern Blot can be done to confirm that the isolated fragments detect regions of differential methylation. Test and control genomic DNA can be cut with a methyl-sensitive enzyme and hypomethylation or hypermethylation at a specific site can be detected by observing whether the size or intensity of a DNA fragment cut with the restriction enzymes is the same between samples. This can be done by electrophoresis analysis and hybridizing the probe to the test and control DNA samples and observing whether the two hybridization complexes are the same or different sizes or intensities. Detailed methodology for gel electrophoretic and nucleic acid hybridization techniques can be found in Sambrook et al., Molecular Biology: A laboratory Approach, Cold Spring Harbor, N.Y. 1989.

The fragment sequences can then be screened for polymorphic markers which can be used as methyl-polymorphic probes as described herein. Probes isolated by the technique described above have at least 14 nucleotides to about 200 nucleotides.

Examples of suitable restriction enzymes for use in the above method include, but are not limited to BsiSI, Hin2I, MseI, Sau3A, RsaI, TspEI, MaeI, NiaIII, DpnI and the like. A preferred methyl-sensitive enzyme is Hpa II that recognizes and cleaves at nonmethylated CCGG sequences but not at CCGG sequences where the outer cytosine is methylated.

Differential methylation can also be assessed by the methods described in U.S. Application No. 2003009997, which discloses a method for detecting the presence of differential methylation between two sources of DNA using enzymes that degrade either unmethylated or methylated DNA. For example, DNA from a healthy individual can be treated with a mixture of methyl-sensitive enzymes that cleave only unmethylated DNA, such as HpaII, HhaI, MaeI, BstUI, and AciI so as to degrade unmethylated DNA. DNA from a lung cancer patient can then be treated with an enzyme that degrades methylated DNA, such as McrBC (New England Biolabs). Subtractive hybridization then permits selective extraction of sequences that are differentially methylated between healthy individuals and individuals with lung cancer.

Alternative methods to detect differential methylation include bisulfide treatment followed by either 1) sequencing, or 2) base-specific cleavage followed by mass spectrometric analysis as described in von Wintzingerode et al., 2002, PNAS, 99.7039-44, herein incorporated by reference.

To serve as a probe, the identified methyl-polymorphic markers can be labeled by any procedure known in the art, for example by incorporation of nucleotides linked to a “reporter molecule” as defined above.

Alternatively, the identified methyl-polymorphic markers need not be labeled and can be used to quantitate allelic frequency using a mass spectrometry technique described in Ding C. and Cantor C. R., 2003, Proc. Natl. Acad. Sci. U.S.A. 100, 3059-64, which is herein incorporated by reference in its entirety.

### Applications

The methods, nucleic acids, and scraping instrument of the present invention can be used in a multitude of applications.

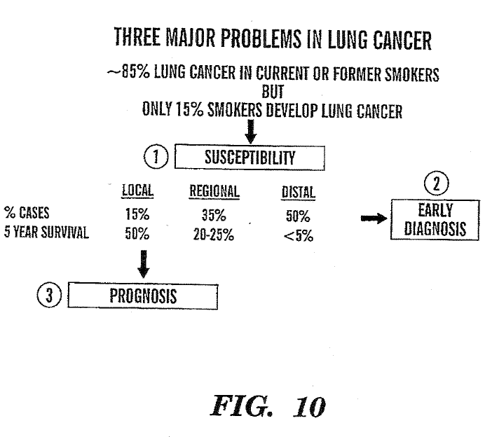

The present invention contemplates identifying a subset of smokers who respond differently to cigarette smoke and appear thus to be predisposed, for example, to its carcinogenic effects, which permits us to screen for individuals at risks of developing lung diseases. As depicted in FIG. 10, lung cancer presents three major problems. While 85% of lung cancer is found in current or former smokers, only 15% of smokers develop lung cancer. A first issue is identifying those individuals who have a susceptibility to develop lung cancer, which is critical to both early diagnosis and prognosis. 15% of lung cancers are diagnoses when the cancer is still highly localized; for these patients, 5 year survival is 50%. However, for the 50% of lung cancer patients diagnosed with distal cancer, 5 year survival is less than 5%. Thus, early diagnosis is critical.

The term “control” or phrases “group of control individuals” or “control individuals” as used herein and throughout the specification refer to at least one individual, preferably at least 2, 3, 4, 5, 6, 7, 8, 9, or 10 individuals, still more preferably at least 10-100 individuals or even 100-1000 individuals, whose airways can be considered having being exposed to similar pollutants than the test individual or the individual whose diagnosis/prognosis/therapy is in question. As a control these are individuals who are selected to be similar to the individuals being tested. For example, if the individual is a smoker, the control group consists of smokers with similar age, race and smoking pattern or pack years of smoking. Whereas if the individual is a non-smoker the control is from a group of non-smokers.

Lung disorders which may be diagnosed or treated by methods described herein include, but are not limited to, asthma, chronic bronchitis, emphysema, bronchiectasis, primary pulmonary hypertension and acute respiratory distress syndrome. The methods described herein may also be used to diagnose or treat lung disorders that involve the immune system including, hypersensitivity pneumonitis, eosinophilic pneumonias, and persistent fungal infections, pulmonary fibrosis, systemic sclerosis, ideopathic pulmonary hemosiderosis, pulmonary alveolar proteinosis, cancers of the lung such as adenocarcinoma, squamous cell carcinoma, small cell and large cell carcinomas, and benign neoplasms of the lung including bronchial adenomas and hamartomas.

One embodiment of the invention provides a method to identify individuals exposed to environmental pollutants, e.g., smokers, who have or are at risk for developing lung cancer, by profiling buccal epithelial cells for the expression of gene(s) associated with different stages of lung cancer.

In one embodiment of the invention, the isolated buccal epithelial cell nucleic acid can be used to develop a diagnostic test for a range of conditions that could be performed in a non-invasive fashion, as a routine screening procedure by scraping cells from the mouth, rather than cells obtained by bronchoscopy. One particularly preferred condition amenable to such diagnosis is lung cancer, including the risk of developing lung cancer.

One embodiment of the invention provides identifying genes which comprise different mouth transcriptomes. One useful mouth transcriptome is comprised of genes which are expressed in the bronchi and whose expression in the bronchi is affected by cigarette smoke, and are also expressed in the mouth. Another useful transcriptome is a lung cancer diagnostic mouth transcriptome. One method for identifying the genes which comprises a lung cancer diagnostic mouth transcriptome is to first identify a mouth transcriptome (as described above), and then determining which of those genes are differentially expressed in the mouth of individuals with lung cancer and healthy individuals.

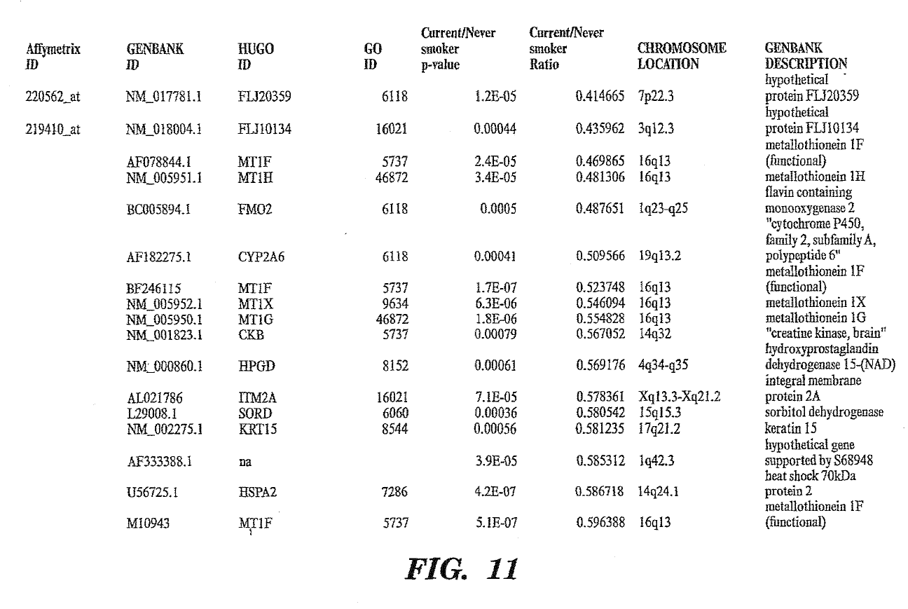

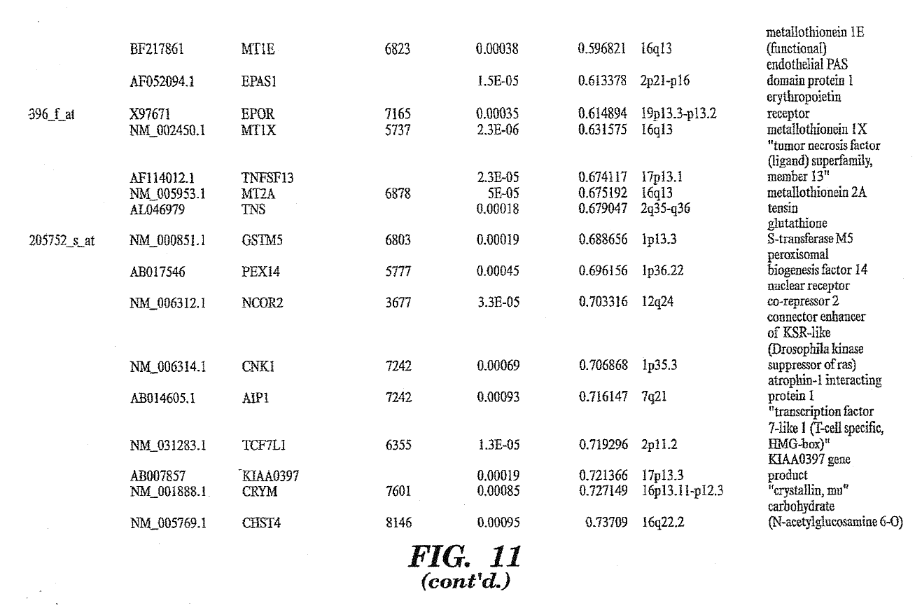

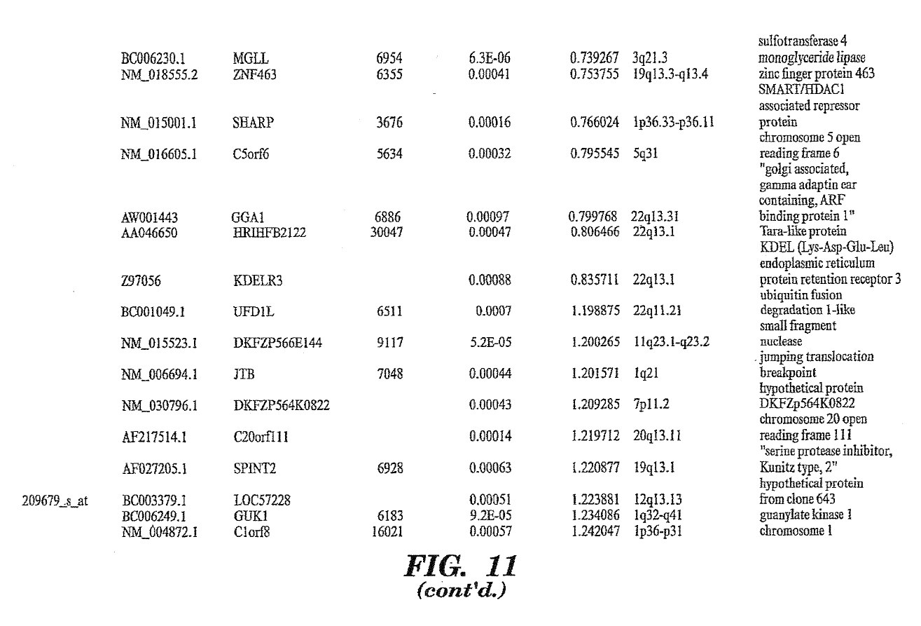

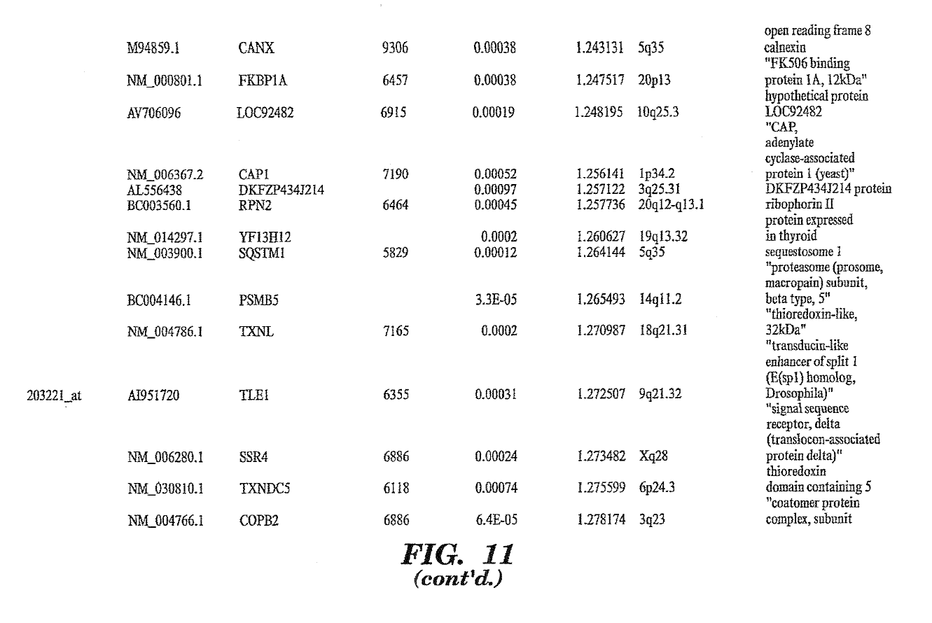

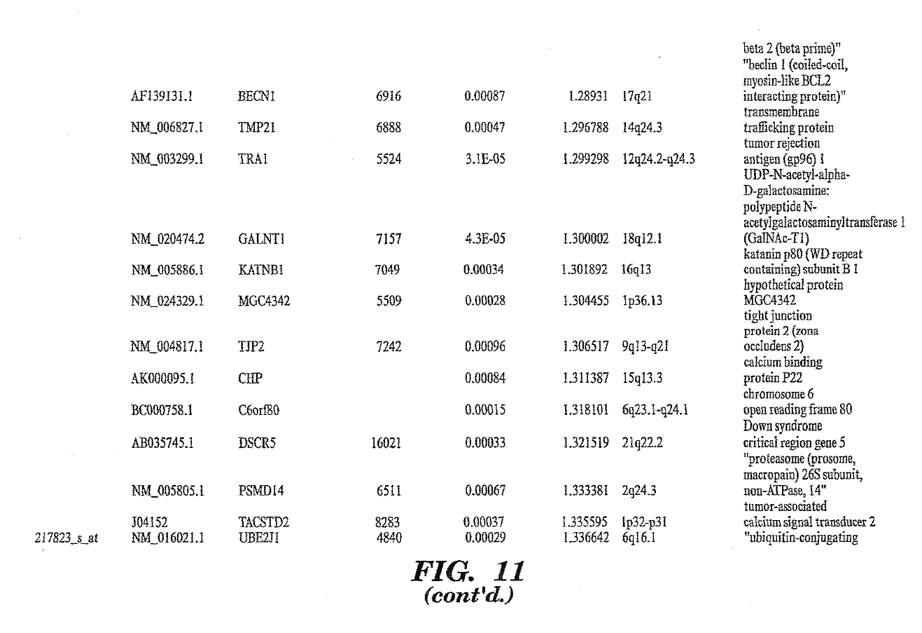

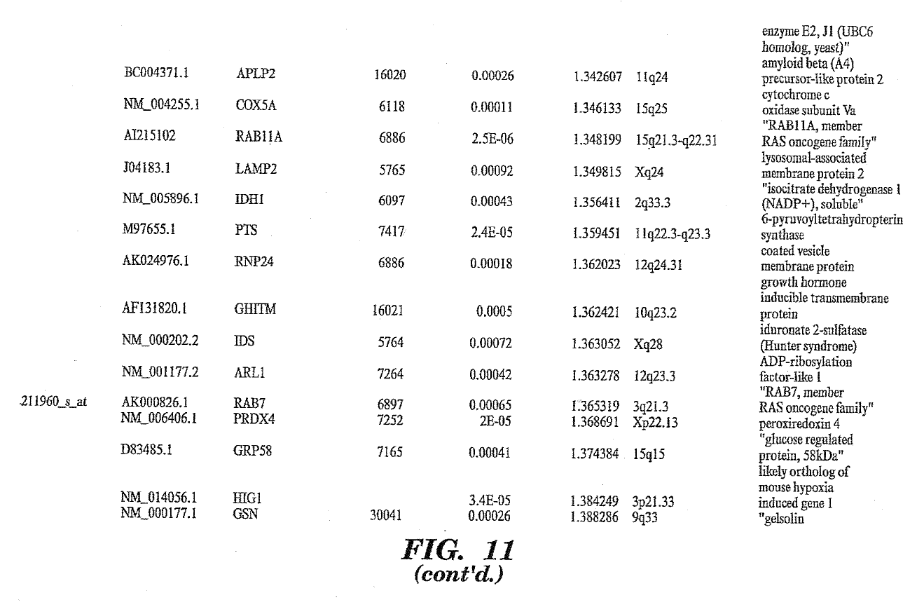

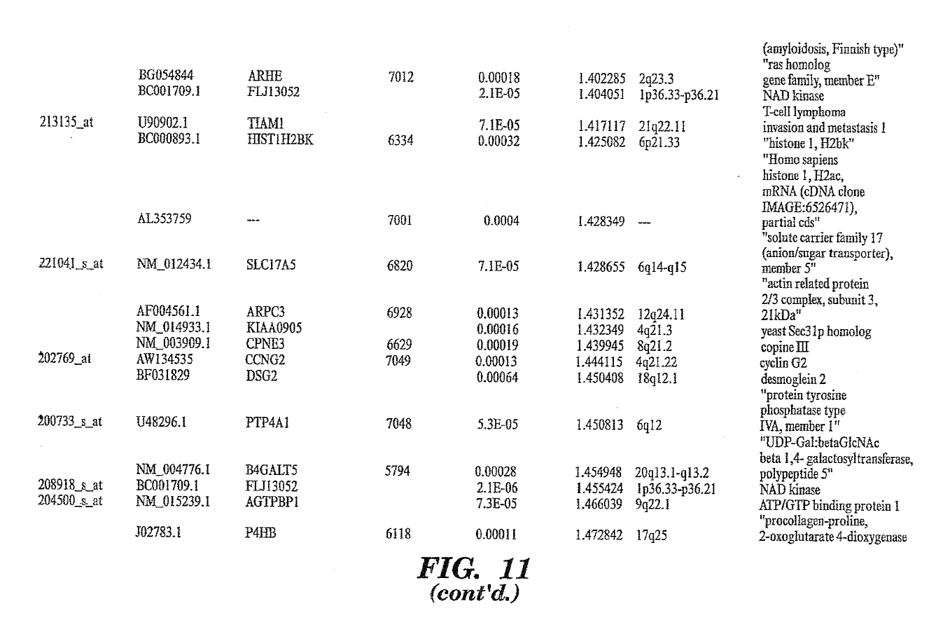

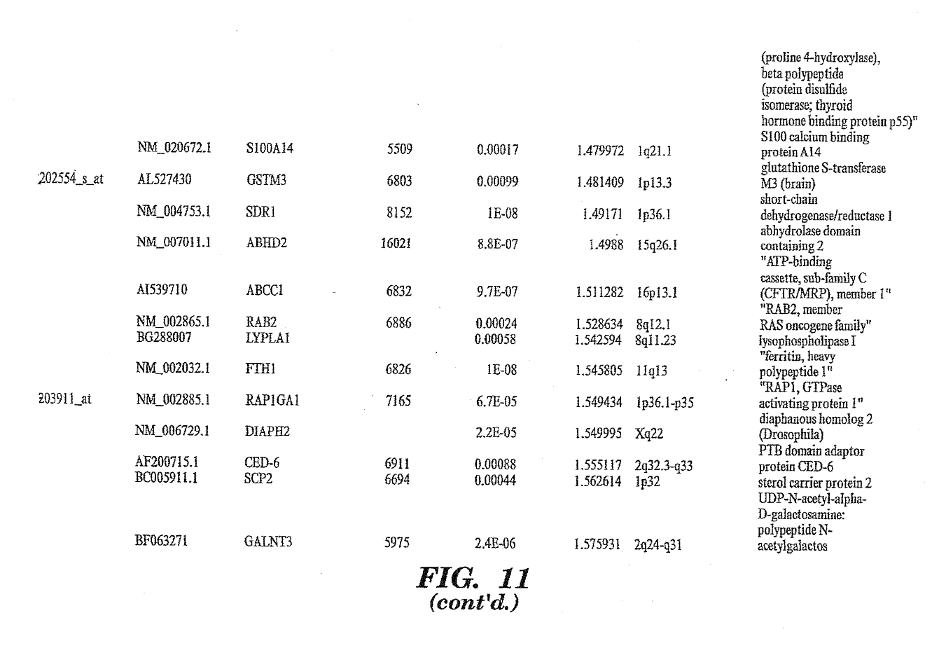

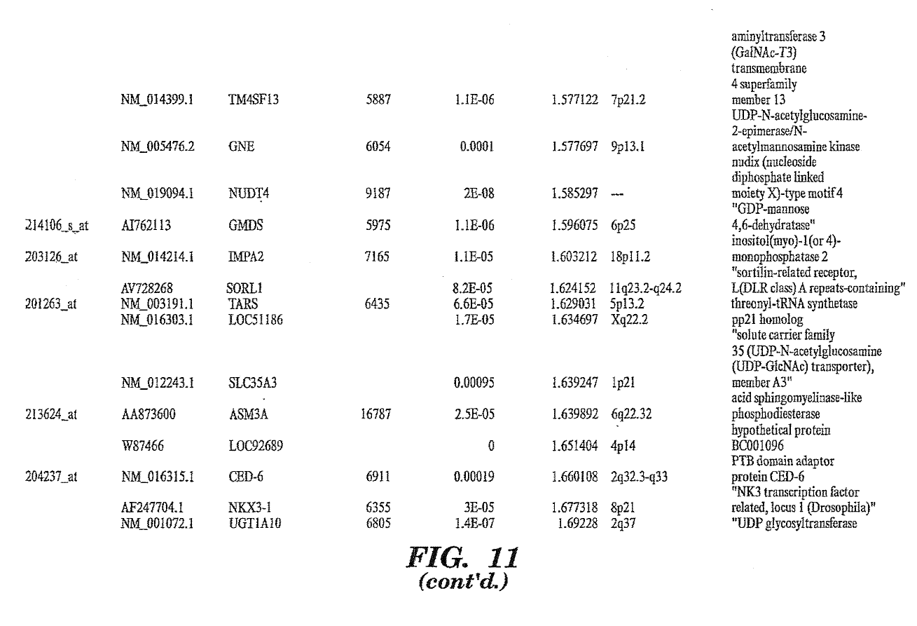

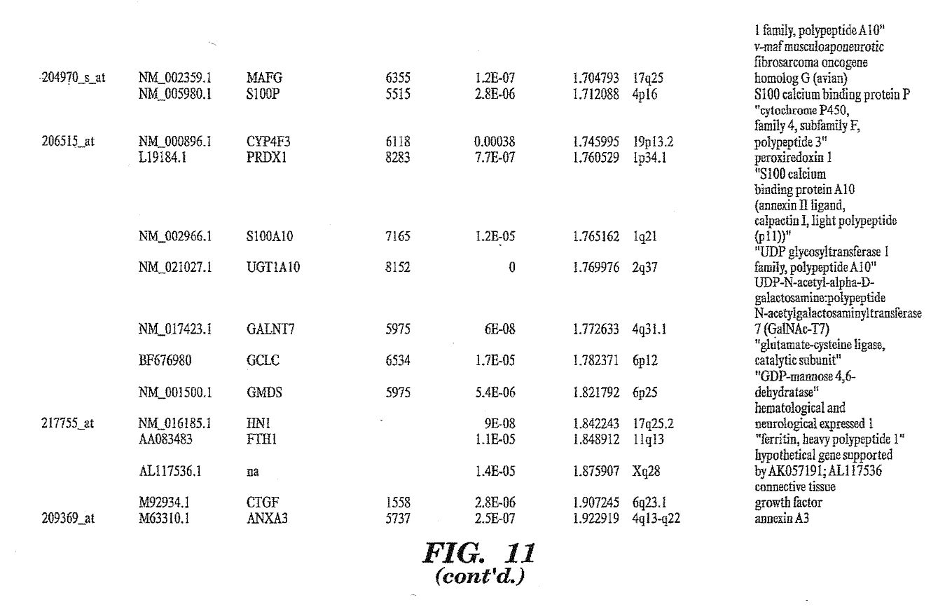

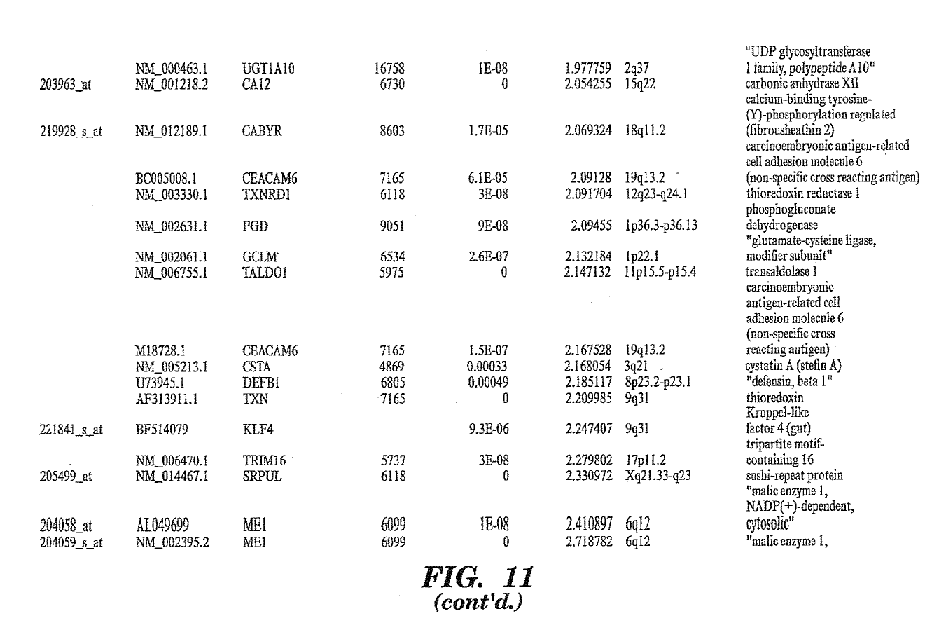

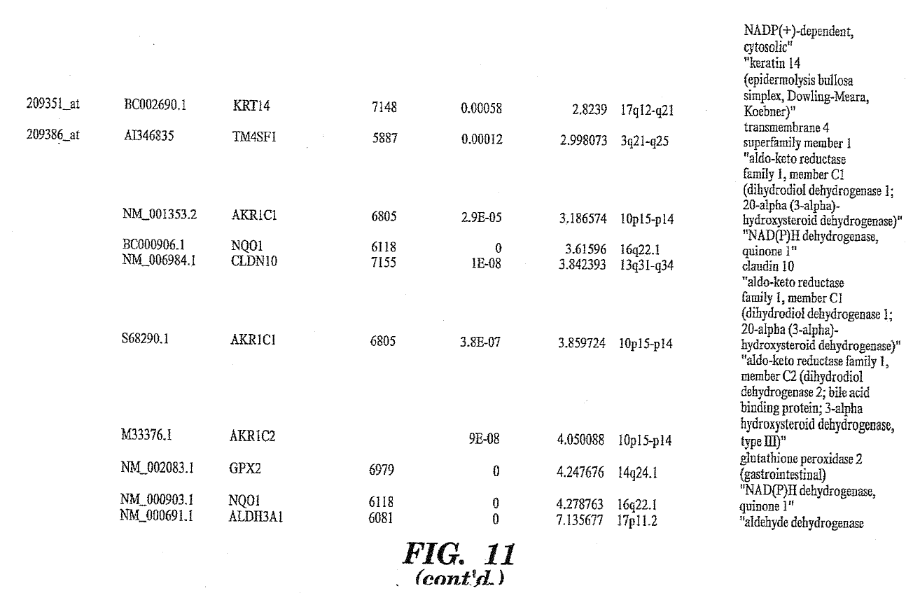

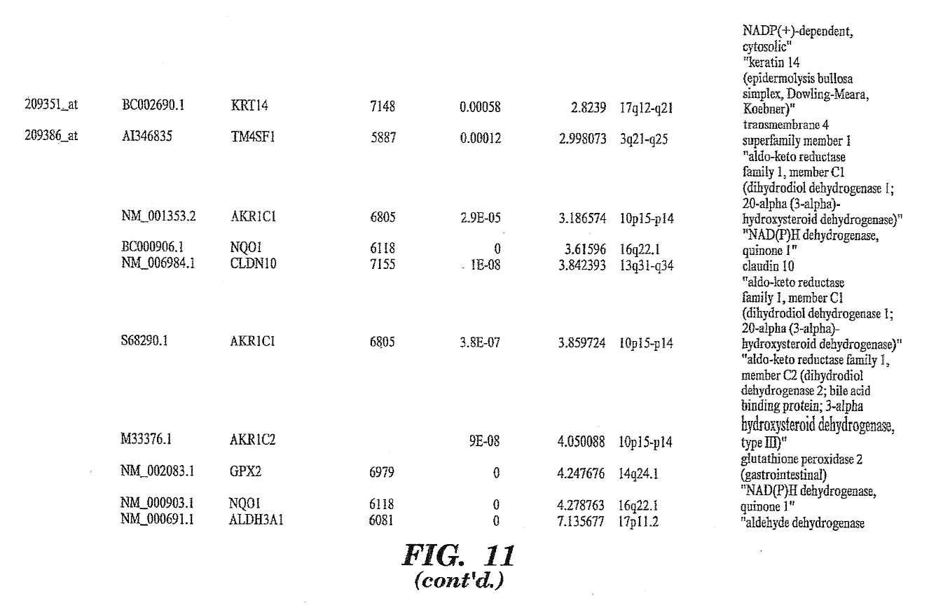

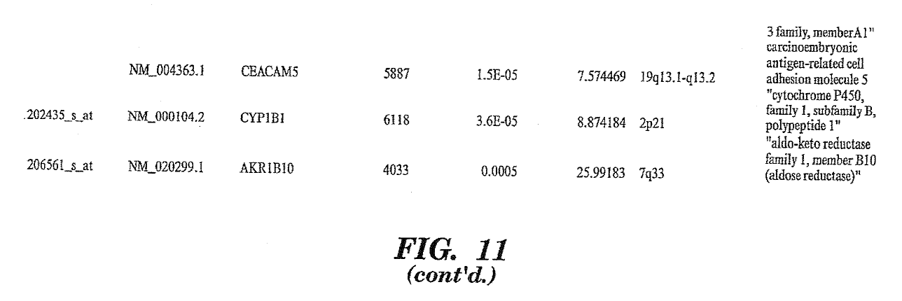

In one embodiment, we have now identified about 166 genes which comprise a mouth transcriptome, i.e, genes which are expressed in the bronchi and whose expression in the bronchi is affected by cigarette smoke, and which are also expressed in the month, consisting of the following genes: ABCC1; ABHD2; AF333388.1; AGTPBP1; AIP1; AKR1B10AKR1C1; AKR1C2; AL117536.1; AL353759; ALDH3A1; ANXA3; APLP2; ARHE; ARL1; ARPC3; ASM3A; B4GALT5; BECN1; C1orf8; C20orf111; C5orf6; C6orf80; CA12; CABYR; CANX; CAP1; CCNG2; CEACAM5; CEACAM6; CED-6; CHP; CHST4; CKB; CLDN10; CNK1; COPB2; COX5A; CPNE3; CRYM; CSTA; CTGF; CYP1B1; CYP2A6; CYP4F3; DEFB1; DIAPH2; DKFZP434J214; DKFZP564K0822; DKFZP566F144; DSCR5; DSG2; EPAS1; EPOR; FKBP1A; FLJ0134; FLJ13052; FLJ130521; FLJ20359; FMO2; FTH1; GALNT1; GALNT3; GALNT7; GCLC; GCLM; GGA1; GHITM; GMDS; GNE; GPX2; GRP58; GSN; GSTM3; GSTM5; GUK1; HIG1; HIST1H2BK; HN1; HPGD; HRIHFB2122; HSPA2; IDH1; IDS; IMPA2; ITM2A; JTB; KATNB1; KDELR3; KIAA0397; KIAA0905; KLF4; KRT14; KRT15; LAMP2; LOC51186; LOC57228; LOC92482; LOC92689; LYPLA1; MAFG; ME1; MGC4342; MGLL; MT1E; MT1F; MT1G; MT1H; MT1X; MT2A; NCOR2; NKX3-1; NQO1; NUDT4; ORL1; P4HB; PEX14; PGD; PRDX1; PRDX4; PSMB5; PSMD14; PTP4A1; PTS; RAB11A; RAB2; RAB7; RAP1GA1; RNP24; RPN2; S100A10; S100A14; S100P; SCP2; SDR1; SHARP1; SLC17A5; SLC35A3; SORD; SPINT2; SQSTM1; SRPUL; SSR4; TACSTD2; TALDO1; TARS; TCF7L1; TIAM1; TJP2; TLE1; TM4SF1; TM4SF13; TMP21; TNFSF13; TNS; TRA1; TRIM16; TXN; TXNDC5; TXNL; TXNRD1; UBE2J1; UFD1L; UGT1A10; YF13H12; and ZNF463. The symbols represent the HUGO identification symbols. FIG. 11 lists details of each of the transcripts corresponding to these genes, including the expression ratio of these genes as compared between smokers and non-smokers (current smoker/never smoker ratio) and the p-value, which shows the significance of the difference in expression of these genes in smokers and non-smokers (current smoker/never smoker p-value). FIG. 11 also shows the gene various gene symbols that these genes appear in databases including HUGO, GenBank and GO databases. Also the Affymetrix cDNA chip location of these transcripts is shown. In one embodiment, the expression of these genes between individuals with lung cancer and healthy individuals is compared, in order to identify genes which form a lung cancer diagnostic mouth transcriptome.

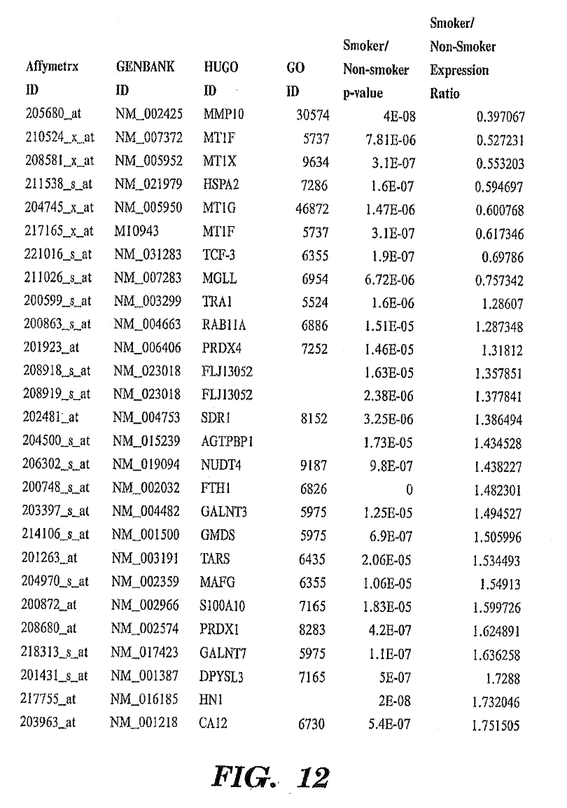

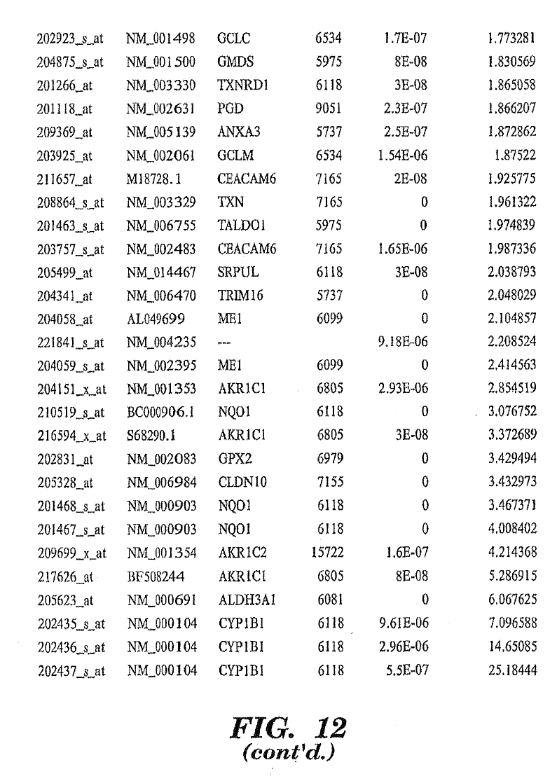

In one preferred embodiment, another mouth transcriptome consists of the following genes, identified using their Human Genome Organization (HUGO) identification symbols: AGTPBP1; AKR1C1; AKR1C2; ALDH3A1; ANXA3; CA12; CEACAM6; CLDN10; CYP1B1; DPYSL3; FLJ13052; FTH1; GALNT3; GALNT7; GCLC; GCLM; GMDS; GPX2; HN1; HSPA2; MAFG; ME1; MGLL; MMP1O; MT1F; MT1G; MT1X; NQO1; NUDT4; PGD; PRDX1; PRDX4; RAB11A; S100A10; SDR1; SRPUL; TALDO1; TARS; TCF-3; TRA1; TRIM16; TXN; and TXNRD1. FIG. 12 lists details of each of the identified transcripts corresponding to these genes including the expression ratio of these genes as compared between smokers and non-smokers (smoker/non-smoker expression ratio) and the p-value, which shows the significance of the difference in expression of these genes in smokers and non-smokers (smoker/non-smoker p-value). In one preferred embodiment, the expression of these genes between individuals with lung cancer and healthy individuals is compared, in order to identify genes which form a lung cancer diagnostic mouth transcriptome.

One preferred embodiment of the invention provides a method to identify “outlier” genes, which can serve as biomarkers for susceptibility to the carcinogenic effects of cigarette smoke and other air pollutants. Such outlier genes are defined as those genes divergently expressed in a small subset of individuals at risk for sat pollutant, e.g. tobacco smoke for smokers who develop lung cancer, and represent a failure of these smokers to mount an appropriate response to cigarette exposure and indicate a linkage to increased risk for developing lung cancer. For example, using the previously described airway transcriptome, we identified a subset of three current smokers who did not upregulate expression of a number of predominantly redox/xenobiotic genes to the same degree as other smokers. One of these smokers developed lung cancer within 6 months of the analysis. In addition, we found a never smoker, who is an outlier among never smokers and expresses a subset of genes at the level of current smokers. These divergent patterns of gene expression in a small subset of smokers represent a failure of these smokers to mount an appropriate response to cigarette exposure and indicate a linkage to increased risk for developing lung cancer.

Therefore, in one embodiment, the invention provides a method of determining an increased risk of lung disease, such as lung cancer, in a smoker comprising taking an airway sample from the individual, analyzing the expression of at least one, preferably at least two, still more preferably at least 4, still more preferably at least 5, still more preferably at least 6, still more preferably at least 7, still more preferably at least 8, still more preferably at least 8, and still more preferably at least all 9 of the outlier genes, wherein deviation of the expression of at least one, preferably at least two, still more preferably at least 4, still more preferably at least 5, still more preferably at least 6, still more preferably at least 7, still more preferably at least 8, still more preferably at least 8, and still more preferably at least all 9 as compared to a control group is indicative of the smoker being at increased risk of developing a lung disease, for example, lung cancer.

In one embodiment of the invention, sufficient nucleic acid from mouth epithelial cells can be obtained to characterize the patterns of expression of over 6,000 genes in different disease states. Preferably, during progressive stages of lung cancer. In this embodiment, the isolated nucleic from epithelial cells can be used to define the normal pattern of gene expression (hereafter called a mouth transcriptome) for different populations, to identify factors such as age, sex, and race that might influence the transcriptome. Similarly, it has already been established that smokers have a profoundly altered pattern of airway epithelial gene expression, and that many of the genes that are altered in current smokers remain abnormal after individuals have stopped smoking. One subset of genes which comprise the airway transcriptome of particular interest is expressed in the mouth, and is referred to herein as the mouth transcriptome.

The isolated nucleic acid of the present invention is also useful to identify genes that are additionally altered in mouth epithelial cells of smokers who have lung cancer, and developing a “class prediction” algorithm to identify smokers with lung cancer.

The divergent patterns of gene expression in a small subset of smokers represent a failure of these smokers to mount an appropriate response to cigarette exposure and indicates a linkage to increased risk for developing lung cancer (Spira et al., 2004). As a result, such target genes can serve as biomarkers for susceptibility to the carcinogenic effects of cigarette smoke and other air pollutants.

Therefore, in one embodiment, the invention provides a method of determining an increased risk of lung disease, such as lung cancer, in a smoker comprising taking a mouth epithelial cells sample from the individual, analyzing the expression of at least one, preferably at least two, still more preferably at least 4, still more preferably at least 5, still more preferably at least 6, still more preferably at least 7, still more preferably at least 8, still more preferably at least 8, and still more preferably at least all of the target genes, wherein genetic alteration of at least one, preferably at least two, still more preferably at least 4, still more preferably at least 5, still more preferably at least 6, still more preferably at least 7, still more preferably at least 8, still more preferably at least 8, and still more preferably at least all 9 as compared to a control group is indicative of the smoker being at increased risk of developing a lung disease, for example, lung cancer.

In one preferred embodiment, the genetic alteration is an increased level of gene expression. In another preferred embodiment, the genetic alteration is a decreased level of gene expression. In one preferred embodiment, the genetic alteration is a deviation in DNA methylation as compared to a healthy individual.

In one particularly preferred embodiment, the isolated RNA can be used for gene expression profiling using a nucleic acid chip based assay to profile many genes at one. For example, using Affymetrix U133 human gene expression arrays.

In another particularly preferred embodiment, the use of the isolated RNA of the present invention can be used to develop a lung cancer diagnostic array.

The methods disclosed herein can also be used to show exposure of a nonsmoker to environmental pollutants by showing increased expression or decreased expression of target genes in a biological sample taken from the mouths of the nonsmokers. If such changes are observed, an entire group of individuals at work or home environment of the exposed individual may be analyzed and if any of them does not show the indicative increases and decreases in the expression of the mouth transcriptome, they may be at greater risk of developing a lung disease and susceptible for intervention. These methods can be used, for example, in a work place screening analyses, wherein the results are useful in assessing working environments, wherein the individuals may be exposed to cigarette smoke, mining fumes, drilling fumes, asbestos and/or other chemical and/or physical airway pollutants. Screening can be used to single out high risk workers from the risky environment to transfer to a less risky environment.

Accordingly, in one embodiment, the invention provides prognostic and diagnostic methods to screen for individuals at risk of developing diseases of the lung, such as lung cancer, comprising screening for changes in the gene expression pattern of the mouth transcriptome. The method comprises obtaining a nucleic acid sample from the mouth of an individual and measuring the level of expression of gene transcripts of the mouth transcriptome as provided herein. Preferably, the level of at least two, still more preferably at least 3, 4, 5, 6, 7, 8, 9, 10 transcripts, and still more preferably, the level of at least 10-15, 15-20, 20-50, or more transcripts, and still snore preferably all of the genes of the mouth transcriptome are measured, wherein difference in the expression of at least one, preferably at least two, still more preferably at least three, and still more preferably at least 4, 5, 6, 7, 8, 9, 10, 10-15, 15-20, 20-30, 30-40, 40-50, 50-60, 60-70, 70-80, 80-85 genes present in the mouth transcriptome compared to a normal mouth transcriptome is indicative of increased risk of a lung disease. The control being at least one, preferably a group of more than one individuals exposed to the same pollutant and having a normal or healthy response to the exposure.

In one embodiment, difference in at least one of the target genes compared to the level of these genes expressed in a control, is indicative of the individual being at an increased risk of developing diseases of the lung.

In one embodiment, the invention provides a prognostic method for lung diseases comprising detecting gene expression changes in at least on of the target genes of the mouth transcriptome, wherein increase in the expression compared with control group is indicative of an increased risk of developing a lung disease.

In one preferred embodiment, the invention provides a tool for screening for changes in the mouth transcriptome during long time intervals, such as weeks, months, or even years. The mouth transcriptome expression analysis is therefore performed at time intervals, preferably two or more time intervals, such as in connection with an annual physical examination, so that the changes in the mouth transcriptome expression pattern can be tracked in individual basis. The screening methods of the invention are useful in following up the response of the airways to a variety of pollutants that the subject is exposed to during extended periods. Such pollutants include direct or indirect exposure to cigarette smoke or other air pollutants.

The methods and scraping instrument of the present invention can be used to study the connection between epithelial cell damage at different parts of the airway with the susceptibility, early diagnosis, and prognosis of lung disorders, including lung cancer. For example, the biomarkers of the present invention can be used on nucleic acid samples from the mouth to determine an individual's susceptibility to developing a lung disorder. Similarly, analysis of the bronchi is useful for early diagnosis, while analysis of the lung tissue itself can relate to prognosis. Such methods are also described in international application PCT/US2004/18460, which is herein incorporated in its entirety.

The methods and scraping instrument of the present invention can be used for epidemiological studies, including assessing the effect of different factors on the development of or risk of development of a lung disorder. Specific factors of interest for such epidemiological studies include but are not limited to racial factors, family genetics, and exposure to second hand smoke.

Similarly, the methods and scraping instrument of the present invention can be used for clinical studies, including address the development of new cigarettes, to assess the effectiveness of different chemoprevention approaches, and the effect of smoking cessation on the development of or risk of development of a lung disorder.

The present invention has many preferred embodiments and relies on many patents, applications and other references for details known to those of the art. Therefore, when a patent, application, or other reference is cited or repeated throughout the specification, it should be understood that it is incorporated by reference in its entirety for all purposes as well as for the proposition that is recited.

### Example

In order to collect intact RNA from buccal mucosal epithelium for studies of the biologic effect of smoking on the airway epithelium, we have developed a relatively non-invasive method for obtaining small amounts of RNA from the mouth. We have measured expression of selected genes in individual subjects using quantitative real time PCR and have used a recently described mass spectrometry method that requires only nanogram amounts of total RNA for analysis and lends itself to high-throughput analysis of hundreds of genes.

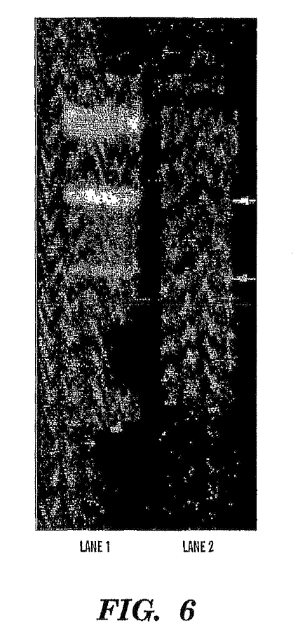

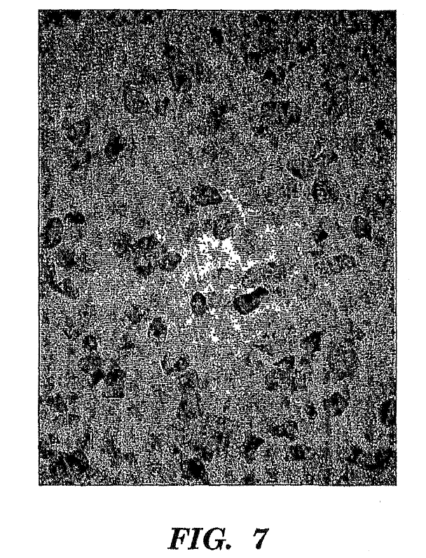

We used micropipette tip cut lengthwise to collect epithelial cells from the buccal mucosa in a relatively noninvasive fashion. We subsequently designed a standardized plastic tool that is concave with serrated edges. It is 5/16 inches wide and 1 6/16 inches long with a 3 inch handle that can be broken off when the scraping tool with collected cells is inserted into a 2 ml microfuge tube containing 1 ml of RNA later solution (Qiagen, Valencia, Calif.). The tool has two features that allow collection of a significant amount of good quality RNA from the buccal mucosa; a finely serrated edge that can scrape off several layers of epithelial cells, and a concave surface that collects the cells. Using gentle pressure, the serrated edge was scraped (ten times) against the buccal mucosa on the inside of the cheek, and cells collected were immediately immersed in 1 cc of RNAlater solution (Qiagen, Valencia, Calif.). After stabilization at 4° C. for up to 24 hours, total RNA from buccal epithelial cells was isolated from the cell pellet using TRIzol reagent (Invitrogen, Carlsbad, Calif.) as per the manufacturer protocol. Integrity of the RNA was confirmed in select cases on an RNA denaturing gel (see FIG. 6). Epithelial cell content was quantified by cytocentrifugation (ThermoShandon Cytospin, Pittsburgh, Pa.) of the cell pellet and staining with a cytokeratin antibody (Signet, Dedham Mass.) (FIG. 7). Using this protocol, we have been able to obtain 300-1500 ng of RNA from each subject (mean+/−standard deviation=983+/−667 ng).

The procedure was well tolerated by all subjects recruited into this study, and none of the subjects experienced bleeding or pain during or after the scrapings. We have tried a number of other instruments including an endoscopic cytobrush (CELEBRITY Endoscopy Cytology Brush, Boston Scientific, Boston, Mass.), cell lifter (Corning Inc., Corning, N.Y.), pap smear kit, and tongue depressor, and have not been able to obtain significant quantities of intact RNA using the above protocol. In addition, we have found that storage of the epithelial cells in RNAlater significantly improves the preservation of RNA integrity as compared with placing the cells directly into TRIzol. We have found that cells can also be preserved in RNAlater at room temperature for up to 24 hours prior to RNA isolation.

In order to assess the biological integrity of the RNA collected from the buccal mucosal cells, we measured the expression of a select number of detoxification related genes that might be expected to be altered by exposure to cigarette smoke⁷ as well as a gene involved in cell adhesion. Using the protocol described above, buccal mucosa RNA was collected from 12 never smokers and 14 current smokers.

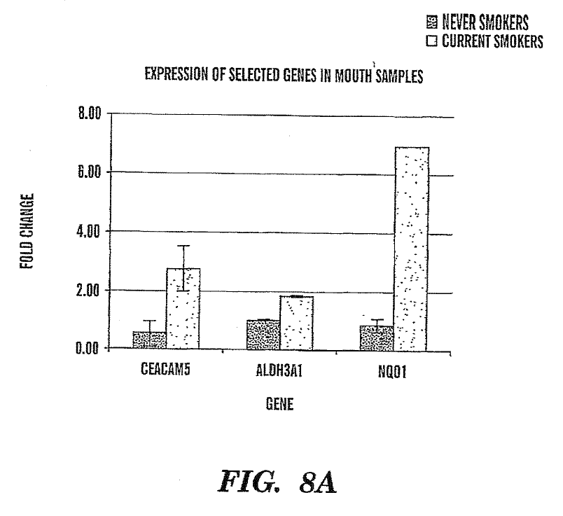

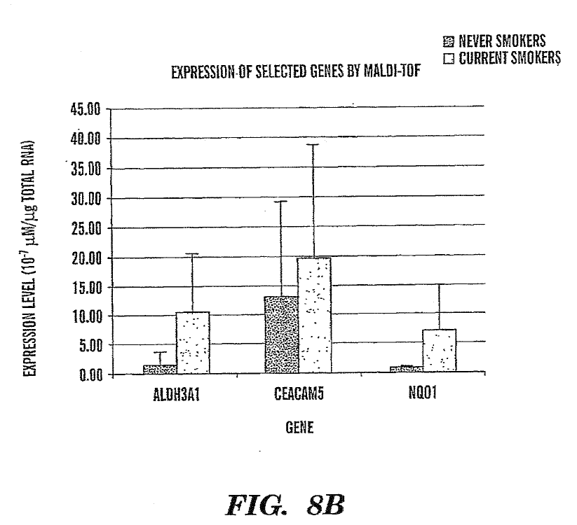

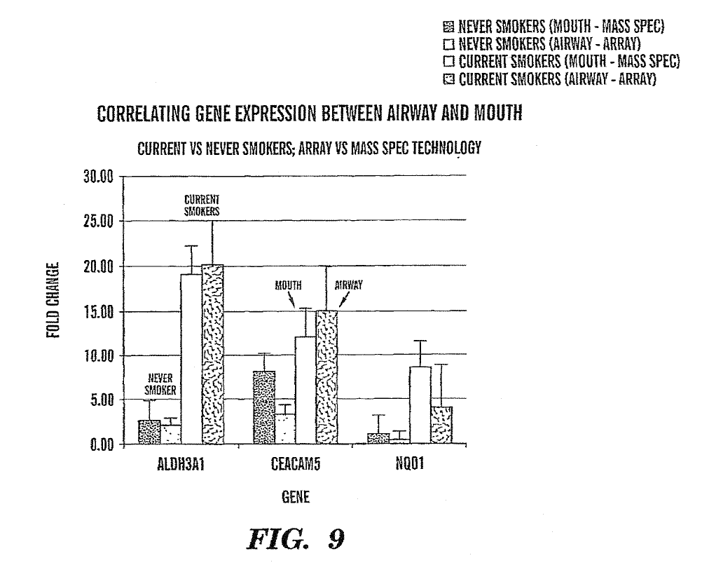

Quantitative real time RT-PCR⁸ was used to measure the expression of NAD(P)H dehydrogenase, quinone 1 (NQO1), aldehyde dehydrogenase family 3, member A1 (ALDH3A1), and carcinoembryonic antigen-related cell adhesion molecule 5 (CEACAM5) from samples obtained from 3 never smokers and 2 current smokers (FIG. 8A and Table 1A). The mean expression of NQO1, ALDH3A1, and CEACAM5 were increased 7, 2 and 3 fold respectively in patients exposed to tobacco smoke. Using competitive PCR and matrix-assisted laser desorption ionization (MALDI) time-of-flight (TOF) mass spectrometry(MS)⁶, we measured the expression of ALDH3A1, NQO1, and CEACAM5 in 7 never smokers and 10 current smokers (FIG. 8B and Table 1B). The expression of all 3 genes was upregulated in smokers compared with never smokers, with statistically significant changes for ALDH3A1 and NQO1.

These studies represent the first successful approach to obtaining RNA from buccal mucosal cells in a non-invasive fashion for measuring gene expression. The method is useful for understanding molecular mechanisms of a variety of diseases that involve the mouth, in assessing the response to and damage caused by inhaled pollutants such as cigarette smoke, the diagnosis and biologic impact of inhaled infections agents, and for developing simple early diagnostic biomarkers of airway and lung cancer that might be applied to screen at-risk populations. The mass spectrometry system allows high-throughput analysis of large numbers of genes (100-200) in short periods of time and could be adapted to mass screening of large numbers of samples.

TABLE 1

Forward and reverse primers for 3 genes
measured by QRT-PCR and MALDI TOF MS.
A. Primers for QRT-PCR

| | |
|---|---|
| ALDH3A1 | 5′-ATG GGA TCC TAC CAT GGC AAG-3′ |
| Forward | [SEQ ID NO: 1] |
| CEACAM5 | 5′-GTC TTG TTT CCC AGA TTT CAG GAA-3′ |
| Forward | [SEQ ID NO: 2] |
| NQO1 | 5′-TGG GAG ACA GCC TCT TAC TTG C-3′ |
| Forward | [SEQ ID NO: 3] |
| ALDH3A1 | 5′-GCG GCG GTG AGA GAA AGT CT-3′ |
| Reverse | [SEQ ID ID: 4] |
| CEACAM5 | 5′-AGA GTG GAT AGC TTA AAA GAA AAA AAG |
| Reverse | TTT C-3′ |
| | [SEQ ID NO: 5] |
| NQO1 | 5′-CAG CTC GGT CCA ATC CCT TC-3′ |
| Reverse | [SEQ ID NO: 6] |

B. Primers for competitive PCR and MALDI-TOF MS

PCR primers

| | |
|---|---|
| ALDH3A1 | 5′-ACGTTGGATGCACTGAAAGAGTTCTACGGG-3′ |
| forward | [SEQ ID NO: 7] |
| CEACAMS | 5′-ACGTTGGATGATGTGAAACCCAGAACCCAG-3′ |
| forward | [SEQ ID NO: 8] |
| NQO1 | 5′-ACGTTGGATGCCACAGAAATGCAGAATGCC-3′ |
| forward | [SEQ ID NO: 9] |
| ALDH3A1 | 5′-ACGTTGGATGCGGGCACTAATGATTCTTCC-3′ |
| reverse | [SEQ ID NO: 10] |
| CEACAM5 | 5′-ACGTTGGATGTCCGGGCCATAGAGGACATT-3′ |
| reverse | [SEQ ID NO: 11] |
| NQO1 | 5′-ACGTTGGATGTGTACTCTCTGCAAGGGATC-3′ |
| reverse | [SEQ ID NO: 12] |

Extension Primers

| | |
|---|---|
| ALDH3A1-E | 5′-GGGAAGATGCTAAGAAATC-3′ |
| | [SEQ ID NO: 13] |
| CEACAM5-E | 5′-CAGGCGCAGTGATTCAGT-3′ |
| | [SEQ ID NO: 14] |
| NQO1-E | 5′-GAATGCCACTCTGAATT-3′ |
| | [SEQ ID NO: 15] |

### REFERENCES

1. King, I. B., J. Satia-Abouta, M. D. Thornquist, J. Bigler, R. E. Patterson, A. R. Kristal, A. L. Shattuck, J. D. Potter, E. White, and J. S. Abouta. 2002. Buccal cell DNA yield, quality, and collection costs: comparison of methods for large-scale studies. Cancer Epidemiol. Biomarkers Prev. 11:1130-1133.
2. Freeman, B., N. Smith, C. Curtis, L. Huckett, J. Mill, and I. W. Craig. 2003. DNA from buccal swabs recruited by mail: evaluation of storage effects on long-term stability and suitability for multiplex polymerase chain reaction genotyping. Behav. Genet. 33:67-72.
3. Bloor, B. K., S. V. Seddon, and P. R. Morgan. 2001. Gene expression of differentiation-specific keratins in oral epithelial dysplasia and squamous cell carcinoma. Oral Oncol. 37:251-261.
4. Loro, L. L., A. C. Johannessen, and O. K. Vintermyr. 2002. Decreased expression of bc1-2 in moderate and severe oral epithelia dysplasias. Oral Oncol. 38:691-698.
5. Ceder, O., J. van Dijken, T. Ericson, and H. Kollberg. 1985. Ribonuclease in different types of saliva from cystic fibrosis patients. Acta Paediatr. Scand. 74:102-106.
6. Ding, C. and C. R. Cantor. 2003. A high-throughput gene expression analysis technique using competitive PCR and matrix-assisted laser desorption ionization time-of-flight MS. Proc. Natl. Acad. Sci. U.S.A 100:3059-3064.
7. Gebel, S., B. Gerstmayer, A. Bosio, H. J. Haussmann, E. Van Miert, and T. Muller. 2003. Gene expression profiling in respiratory tissues from rats exposed to mainstream cigarette smoke. Carcinogenesis.
8. Powell, C. A., A. Spira, A. Derti, C. DeLisi, G. Liu, A. Borczuk, S. Busch, S. Sahasrabudhe, Y. D. Chen, D. Sugarbaker, R. Bueno, W. G. Richards, and J. S. Brody. 2003. Gene expression in lung adenocarciniomas of smokers and nonsmokers. American Journal of Respiratory Cell and Molecular Biology 29:157-162.
   All references described herein are incorporated by reference.

We claim:

1. A method of determining whether an individual is at increased risk of developing a lung disease, comprising: a) taking a biological sample from the mouth of an individual exposed to an airway pollutant or at risk of being exposed to an airway pollutant; and b) analyzing whether there is a genetic alteration in at least one gene of the mouth transcriptome genes of the group consisting of ABCC1; ABHD2; AF33338.1; AGTPBP1; AIP1; AKR1B10AKR1C1; AKR1C2; AL117536.1; AL353759; ALDH3A1; ANXA3; APLP2; ARHE; ARL1; ARPC3; ASM3A; B4GALT5; BECN1; C1orf8; C20orf111; C5orf6; C6orf80; CA12; CABYR; CANX; CAP1; CCNG2; CEACAM5; CEACAM6; CED-6; CHP; CHST4; CKB; CLDN10; CNK1; COPB2; COX5A; CPNE3; CRYM; CSTA; CTGF; CYP1B1; CYP2A6; CYP4F3; DEFB1; DIAPH2; DKFZP434J214; DKFZP564K0822; DKFZP566E144; DSCR5; DSG2; EPAS1; EPOR; FKBP1A; FLJ10134; FLJ13052; FLJ130521; FLJ20359; FMO2; FTH1; GALNT1; GALNT3; GALNT7; GCLC; GCLM; GGA1; GHITM; GMDS; GNE; GPX2; GRP58; GSN; GSTM3; GSTM5; GUK1; HIG1; HIST1H2BK; HN1; HPGD; HRIHFB2122; HSPA2; IDH1; IDS; IMPA2; ITM2A; JTB; KATNB1; KDELR3; KIAA0397; KIAA0905; KLF4; KRT14; KRT15; LAMP2; LOC51186; LOC57228; LOC92482; LOC92689; LYPLA1; MAFG; ME1; MGC4342; MGLL; MT1E; MT1F; MT1G; MT1H; MT1X; MT2A; NCOR2; NKX3-1; NQO1; NUDT4; ORL1; P4HB; PEX14; PGD; PRDX1; PRDX4; PSMB5; PSMD14; PTP4A1; PTS; RAB11A; RAB2; RAB7; RAP1GA1; RNP24; RPN2; S100A10; S100A14; S100P; SCP2; SDR1; SHARP1; SLC17A5; SLC35A3; SORD; SPINT2; SQSTM1; SRPUL; SSR4; TACSTD2; TALDO1; TARS; TCF7L1; TIAM1; TJP2; TLE1; TM4SF1; TM4SF13; TMP21; TNFSF13; TNS; TRA1; TRIM16; TXN; TXNDC5; TXNL; TXNRD1; UBE2J1; UFD1L; UGT1A10; YF13H12; and ZNF463, wherein the presence of a genetic alteration in one or more of the mouth transcriptome genes as compared to the same at least one gene in a group of control individuals is indicative that the individual has an increased risk of developing a lung disease.

2. The method of claim 1, wherein the genetic alteration is selected from the group consisting of deviation of a gene's DNA methylation pattern and deviation of a gene's expression pattern.

3. The method of claim 1, wherein the genetic alteration is a deviation of a gene's expression pattern.

4. The method of claim 1, wherein the airway pollutant is smoke from a cigarette or a cigar and the lung disease is lung cancer.

5. The method of claim 4, wherein the lung cancer is selected from adenocarcinoma, squamous cell carcinoma, small cell carcinoma, large cell carcinoma, and benign neoplasms of the lung.

6. The method of claim 1, wherein the individual is a smoker and one looks at expression of at least one gene selected from the group consisting of mouth transcriptome genes, wherein lower expression of that at least one gene in the smoker than in a control group of corresponding smokers is indicative of an increased risk of developing lung cancer.

7. The method of claim 6, wherein lower expression of at least three genes of the mouth transcriptome is indicative of an increased risk of developing lung cancer.

8. A method of diagnosing predisposition of a smoker to lung disease comprising analyzing an expression pattern of one or more genes selected from the group consisting of ABCC1; ABHD2; AF333388.1; AGTPBP1; AIP1; AKR1B10AKR1C1; AKR1C2; AL117536.1; AL353759; ALDH3A1; ANXA3; APLP2; ARHE; ARL1; ARPC3; ASM3A; B4GALT5; BECN1; C1orf8; C20orf111; C5orf6; C6orf80; CA12; CABYR; CANX; CAP1; CCNG2; CEACAM5; CEACAM6; CED-6; CHP; CHST4; CKB; CLDN10; CNK1; COPB2; COX5A; CPNE3; CRYM; CSTA; CTGF; CYP1B1; CYP2A6; CYP4F3; DEFB1; DIAPH2; DKFZP434J214; DKFZP564K0822; DKFZP566E144; DSCR5; DSG2; EPAS1; EPOR; FKBP1A; FLJ10134; FLJ13052; FLJ130521; FLJ20359; FMO2; FTH1; GALNT1; GALNT3; GALNT7; GCLC; GCLM; GGA1; GHITM; GMDS; GNE; GPX2; GRP58; GSN; GSTM3; GSTM5; GUK1; HIG1; HIST1H2BK; HN1; HPGD; HRIHFB2122; HSPA2; IDH1; IDS; IMPA2; ITM2A; JTB; KATNB1; KDELR3; KIAA0397; KIAA0905; KLF4; KRT14; KRT15; LAMP2; LOC51186; LOC57228; LOC92482; LOC92689; LYPLA1; MAFG; ME1; MGC4342; MGLL; MT1E; MT1F; MT1G; MT1H; MT1X; MT2A; NCOR2; NKX3-1; NQO1; NUDT4; ORL1; P4HB; PEX14; PGD; PRDX1; PRDX4; PSMB5; PSMD14; PTP4A1; PTS; RAB11A; RAB2; RAB7; RAP1GA1; RNP24; RPN2; S100A10; S100A14; S100P; SCP2; SDR1; SHARP1; SLC17A5; SLC35A3; SORD; SPINT2; SQSTM1; SRPUL; SSR4; TACSTD2; TALDO1; TARS; TCF7L1; TIAM1; TJP2; TLE1; TM4SF1; TM4SF13; TMP21; TNFSF13; TNS; TRA1; TRIM16; TXN; TXNDC5; TXNL; TXNRD1; UBE2J1; UFD1L; UGT1A10; YF13H12; and ZNF463 in a biological sample taken from the mouth of the smoker, wherein a divergent expression pattern of one or more of these genes as compared to the expression pattern of these genes in group of control individuals is indicative of the predisposition of the individual to lung disease.

9. The method of claim 8, comprising analyzing an expression pattern of one or more genes selected from the group consisting of AGTPBP1; AKR1C1; AKR1C2; ALDH3A1; ANXA3; CA12; CEACAM6; CLDN10; CYP1B1; DPYSL3; FLJ13052; FTH1; GALNT3; GALNT7; GCLC; GCLM; GMDS; GPX2; HN1; HSPA2; MAFG; ME1; MGLL; MMP10; MT1F; MT1G; MT1X; NQO1; NUDT4; PGD; PRDX1; PRDX4; RAB11A; S100A10; SDR1; SRPUL; TALDO1; TARS; TCF-3; TRA1; TRIM16; and TXN.

10. The method of claim 8, wherein the biological sample is a nucleic acid sample.

11. The method of claim 10, wherein the nucleic acid is RNA.

12. The method of claim 10, wherein the analysis is performed using a nucleic acid array.

13. The method of claim 10, wherein the analysis is performed using quantitative real time PCR or mass spectrometry.
